(19) United States
(12) Patent Application Publication
(10) Pub. No.: US 2023/0058389 A1
(43) Pub. Date: Feb. 23, 2023

(54) DATA INFERENCES FROM A WEARABLE ROBOT

(71) Applicant: ROAM ROBOTICS INC., San Francisco, CA (US)

(72) Inventors: Kyle Kaveny, San Francisco, CA (US); Timothy Alan Swift, Walnut Creek, CA (US); Kyle Motter, Fort Myers, FL (US)

(21) Appl. No.: 17/889,603

(22) Filed: Aug. 17, 2022

Related U.S. Application Data
(60) Provisional application No. 63/234,008, filed on Aug. 17, 2021.

(51) Int. Cl. B25J 9/00; B25J 9/14; B25J 13/08; B25J 13/00; A61B 5/11; A61B 5/00; G01W 1/10

(52) U.S. Cl. CPC B25J 9/0006; B25J 9/146; B25J 13/088; B25J 13/006; A61B 5/1117; A61B 5/1118; A61B 5/742; G01W 1/10; G01W 2203/00

(57) ABSTRACT

A method of operating an exoskeleton system that includes obtaining at an exoskeleton device, sensor data from one or more sensors; and determining, by the exoskeleton device based at least in part on the sensor data, one or more states, including one or more of: at least one state of the exoskeleton system; at least one state of a user wearing the exoskeleton system; and at least one state of a location where the user and exoskeleton system are located. The method further includes determining, by the exoskeleton device, a response based at least in part on the determined one or more states; and generating the response by the exoskeleton device causing actuation of the exoskeleton system.

### CROSS-REFERENCE TO RELATED APPLICATIONS

This application is a non-provisional of and claims the benefit of U.S. Provisional Application No. 63/234,008, filed Aug. 17, 2021, entitled “DATA INFERENCES FROM A WEARABLE ROBOT,” with attorney docket number 0110496-023PR0. This application is hereby incorporated herein by reference in its entirety and for all purposes.

This application is also related to U.S. patent application Ser. No. 17/329,632, filed May 25, 2021, entitled “DIRECT DRIVE PNEUMATIC TRANSMISSION FOR A MOBILE ROBOT,” with attorney docket number 0110496-009US0; and is related to U.S. patent application Ser. No. 17/332,818, filed May 27, 2021, entitled “POWERED MEDICAL DEVICE AND METHODS FOR IMPROVED USER MOBILITY AND TREATMENT,” with attorney docket number 0110496-010US0; and is related to U.S. patent application Ser. No. 17/331,956, filed May 27, 2021, entitled “FIT AND SUSPENSION SYSTEMS AND METHODS FOR A MOBILE ROBOT,” with attorney docket number 0110496-012US0; and is related to U.S. patent application Ser. No. 17/331,961, filed May 27, 2021, entitled “BATTERY SYSTEMS AND METHODS FOR A MOBILE ROBOT,” with attorney docket number 0110496-013US0; and is related to U.S. patent application Ser. No. 17/332,203, filed May 27, 2021, entitled “CONTROL SYSTEM AND METHOD FOR A MOBILE ROBOT,” with attorney docket number 0110496-014US0; and is related to U.S. patent application Ser. No. 17/332,172, filed May 27, 2021, entitled “USER INTERFACE AND FEEDBACK SYSTEMS AND METHODS FOR A MOBILE ROBOT,” with attorney docket number 0110496-015US0; and is related to U.S. patent application Ser. No. 17/332,507, filed May 27, 2021, entitled “DATA LOGGING AND THIRD-PARTY ADMINISTRATION OF A MOBILE ROBOT,” with attorney docket number 00110496-016 US0; and is related to U.S. patent application Ser. No. 17/332,860, filed May 27, 2021, entitled 0110496-017US0, these applications are hereby incorporated herein by reference in their entirety for all purposes.

This application is also related to U.S. Non-Provisional Application No. XXX/YYY,ZZZ, filed contemporaneously herewith, entitled “ACTUATOR FEATURES TO IMPROVE FUNCTION OF A MOBILE ROBOT,” with attorney docket number 0110496-018US0; is related to U.S. Non-Provisional Application No. XXX/YYY,ZZZ, filed contemporaneously herewith, entitled “CABLE MANAGEMENT SYSTEMS AND METHODS FOR A WEARABLE MOBILE,” with attorney docket number 0110496-019US0; is related to U.S. Non-Provisional Application No. XXX/YYY,ZZZ, filed contemporaneously herewith, entitled “MOBILE POWER SOURCE FOR A MOBILE ROBOT,” with attorney docket number 0110496-020US0; is related to U.S. Non-Provisional Application No. XXX/YYY,ZZZ, filed contemporaneously herewith, entitled “UNIFIED PNEUMATIC AND ELECTRICAL CONNECTOR SYSTEM AND METHOD,” with attorney docket number 0110496-021US0; and is related to U.S. Non-Provisional Application No. XXX/YYY,ZZZ, filed contemporaneously herewith, entitled “MARITIME APPLICATIONS FOR A MOBILE ROBOT,” with attorney docket number 0110496-022US0. These applications are hereby incorporated herein by reference in their entirety and for all purposes.

### BRIEF DESCRIPTION OF THE DRAWINGS

FIG. 1 is an example illustration of an embodiment of an exoskeleton system being worn by a user.

FIG. 2 is a front view of an embodiment of a leg actuation unit coupled to one leg of a user.

FIG. 3 is a side view of the leg actuation unit of FIG. 3 coupled to the leg of the user.

FIG. 4 is a perspective view of the leg actuation unit of FIGS. 3 and 4.

FIG. 5 is a block diagram illustrating an example embodiment of an exoskeleton system.

FIG. 6 is a rear view of another embodiment of an exoskeleton system including a leg actuator unit coupled to the right leg of a user.

FIG. 7 is a close-up view of a portion of the illustration of FIG. 6.

FIG. 8 illustrates an embodiment of an exoskeleton network that includes an exoskeleton system that is operably coupled to an external device and a vehicle via a direct connection and/or via a network.

FIG. 9 illustrates another embodiment of an exoskeleton network that comprises a plurality of exoskeleton systems being worn by a plurality of respective users, with the plurality of exoskeletons being operably connected to an exoskeleton server and an admin device via a network.

FIG. 10a illustrates a side view of a pneumatic actuator in a compressed configuration in accordance with one embodiment.

FIG. 10b illustrates a side view of the pneumatic actuator of FIG. 10a in an expanded configuration.

FIG. 11a illustrates a cross-sectional side view of a pneumatic actuator in a compressed configuration in accordance with another embodiment.

FIG. 11b illustrates a cross-sectional side view of the pneumatic actuator of FIG. 11a in an expanded configuration.

FIG. 12a illustrates a top view of a pneumatic actuator in a compressed configuration in accordance with another embodiment.

FIG. 12b illustrates a top view of the pneumatic actuator of FIG. 12a in an expanded configuration.

FIG. 13 illustrates a top view of a pneumatic actuator constraint rib in accordance with an embodiment.

FIG. 14a illustrates a cross-sectional view of a pneumatic actuator bellows in accordance with another embodiment.

FIG. 14b illustrates a side view of the pneumatic actuator of FIG. 14a in an expanded configuration showing the cross section of FIG. 14a.

FIG. 15 illustrates an example planar material that is substantially inextensible along one or more plane axes of the planar material while being flexible in other directions.

It should be noted that the figures are not drawn to scale and that elements of similar structures or functions are generally represented by like reference numerals for illustrative purposes throughout the figures. It also should be noted that the figures are only intended to facilitate the description of the preferred embodiments. The figures do not illustrate every aspect of the described embodiments and do not limit the scope of the present disclosure.

### DETAILED DESCRIPTION OF THE PREFERRED EMBODIMENTS

The following disclosure includes example embodiments of the design of novel exoskeleton devices. Various preferred embodiments include: a leg brace with integrated actuation, a mobile power source and a control unit that determines the output behavior of the device in real-time. In various embodiments, a brace uses a fluidic actuator to help the user move. In order to function, in some examples the brace needs a power and fluid supply and the ability to communicate to receive and send data. In one embodiment, the system can comprise, consist essentially of or consist of a power pack strapped onto the torso of the user and one or more braces worn around the knee(s) of the user. These two components can be connected together in various embodiments so that the brace may communicate with the power pack and/or receive power and fluid from the power pack. This is done in some examples through the use of power and fluidic cables on both components.

Wearable robot and exoskeleton systems of various embodiments can collect data about the user, the environment, and the system. This data may come from a number of different sources, such as but not limited to: sensors directly integrated with the wearable robot system worn by a user, sensors on other wearable robot systems worn by other users, sensors from vehicles, data sent to the wearable robot system wirelessly or through a wire, and the like. The data in some examples can also include any possible statistical measures applied to the data, such as distribution, duration, range, variability, minimum, maximum, median, mean, kurtosis, and the like, as well as any analyses of the data, including kinematic, static, and dynamic calculations of the user's physical orientation and motion. This data can be used in various embodiments to make inferences about any combination of the states of the user, the states of the environment, and the states of the system. Such inferences may be about the past, present, and possible future states of the user, environment and/or system. States of the user can include, but are not limited to, the cognitive, mental, emotional, and/or physical state of the user. The physical state of the user can include, but is not limited to, the user's physical attributes, health metrics, vital signs, exertion level, ability, skill at an activity, as well as kinematic and dynamic information such as whether the user is running, walking, sleeping, crouching, lying down, and the like. States of the environment can include, but are not limited to, weather, terrain, and the presence of animals, people, and structures, both natural and manmade. States of the system can include, but are not limited to, operation, performance, component malfunction, wear and failure.

Sensors in various embodiments can provide information regarding various parameters about the user, the environment or the system, including but not limited to: external influences on the user such as forces, impacts, and the like; the position, velocity, acceleration, and orientation of a user or any part of the user's body, clothing, backpack, other body worn elements, other users and their wearables, and the like; the physical state of the user such as body temperature, muscle fatigue, sweating, blood pressure, heart rate, and the like; the surrounding weather, temperature, pressure, humidity, wind speeds, presence of rain, presence of thunder, presence of snow, altitude changes, steepness or undulation of the terrain, location on Earth, and the like. Some examples of these sensors can include but are not limited to GPS, pressure sensors, force sensors, temperature sensors, accelerometers, gyroscopes, LiDAR, motion sensors, distance sensors, magnetometers, imaging sensors, light sensors, hygrometers, anemometers, barometers, voltage sensors, current sensors, heart rate monitors, electromyography (EMG), and the like. Any single sensor or combination of sensors included with the wearable robotic system and other data sources could provide information about any combination of the state of the user, environment and robotic system in some embodiments. As discussed herein data inferences can be made by an exoskeleton system itself, by a device external and associated with the exoskeleton system (e.g., a smartphone) by a computing system of a vehicle associated with the exoskeleton system, or by a remote device such as an administrator device or an exoskeleton server.

A component of an exoskeleton system that is present in various embodiments is a body-worn, lower-extremity brace that incorporates the ability to introduce torque to the user. One preferred embodiment of this component is a leg brace that is configured to support the knee of the user and includes actuation across the knee joint to provide assistance torques in the extension direction. This embodiment can connect to the user through a series of attachments including one on the boot, below the knee, and along the user's thigh. This preferred embodiment can include this type of leg brace on both legs of the user.

The present disclosure teaches example embodiments of a fluidic exoskeleton system that includes one or more adjustable fluidic actuators. Some preferred embodiments include a fluidic actuator that can be operated at various pressure levels with a large stroke length in a configuration that can be oriented with a joint on a human body.

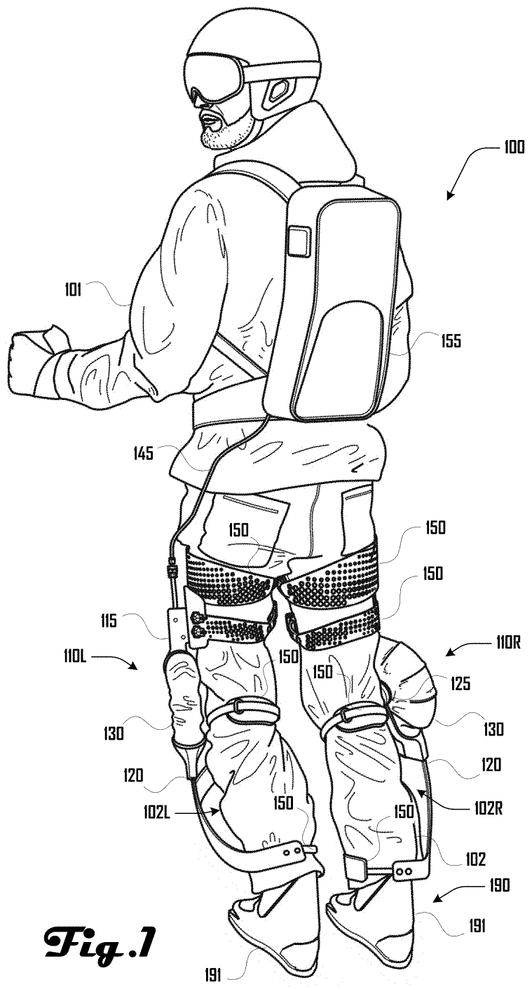

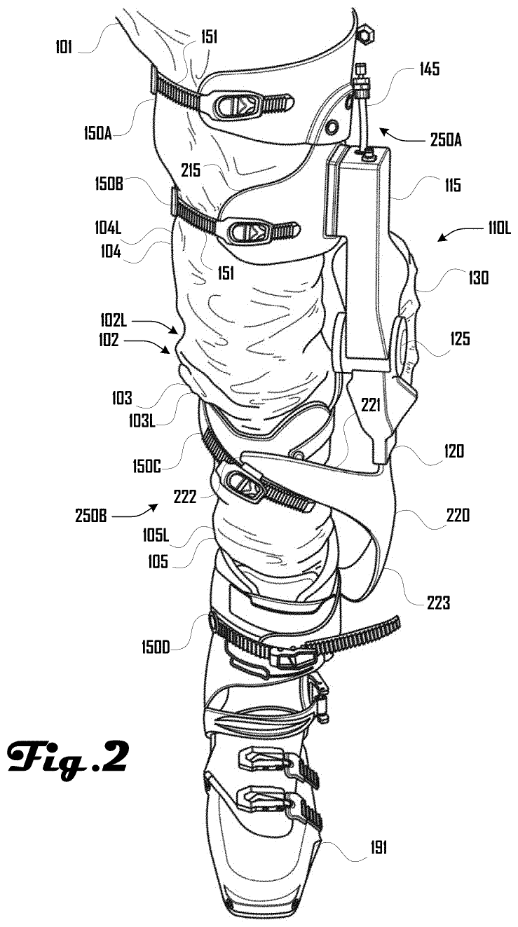

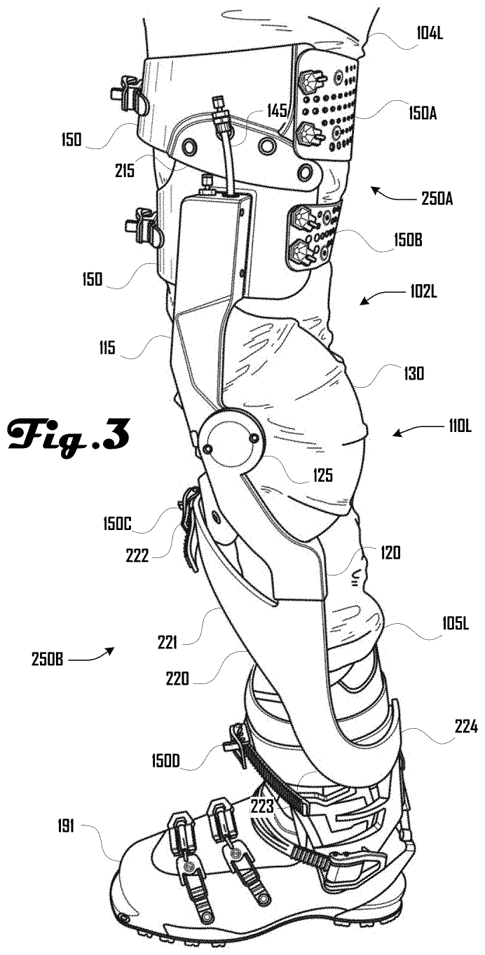

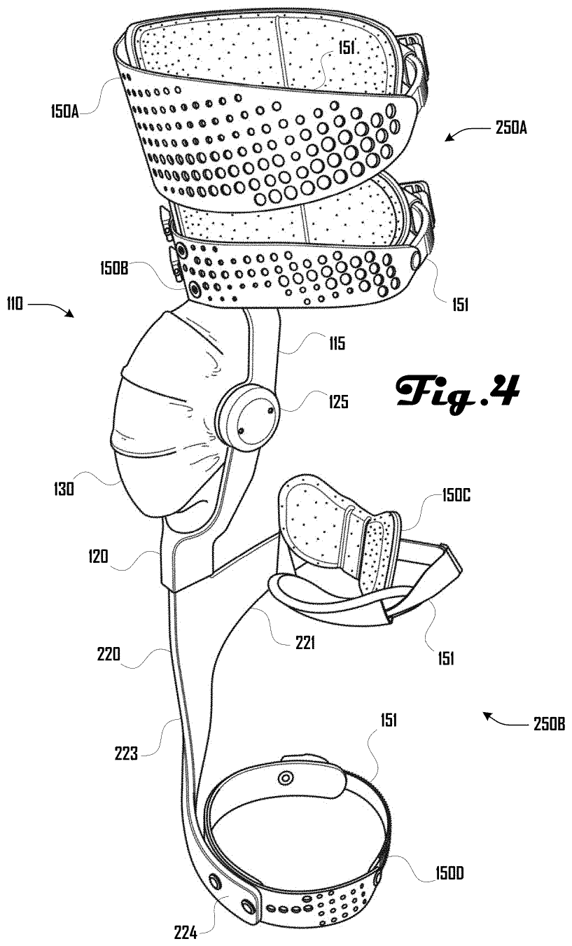

As discussed herein, an exoskeleton system 100 can be configured for various suitable uses. For example, FIGS. 1-3 illustrate an exoskeleton system 100 being used by a user. As shown in FIG. 1 the user 101 can wear the exoskeleton system 100 on both legs 102. FIGS. 2 and 3 illustrate a front and side view of an actuator unit 110 coupled to a leg 102 of a user 101 and FIG. 4 illustrates a side view of an actuator unit 110 not being worn by a user 101.

As shown in the example of FIG. 1, the exoskeleton system 100 can comprise a left and right leg actuator unit 110L, 110R that are respectively coupled to a left and right leg 102L, 102R of the user. In various embodiments, the left and right leg actuator units 110L, 110R can be substantially mirror images of each other.

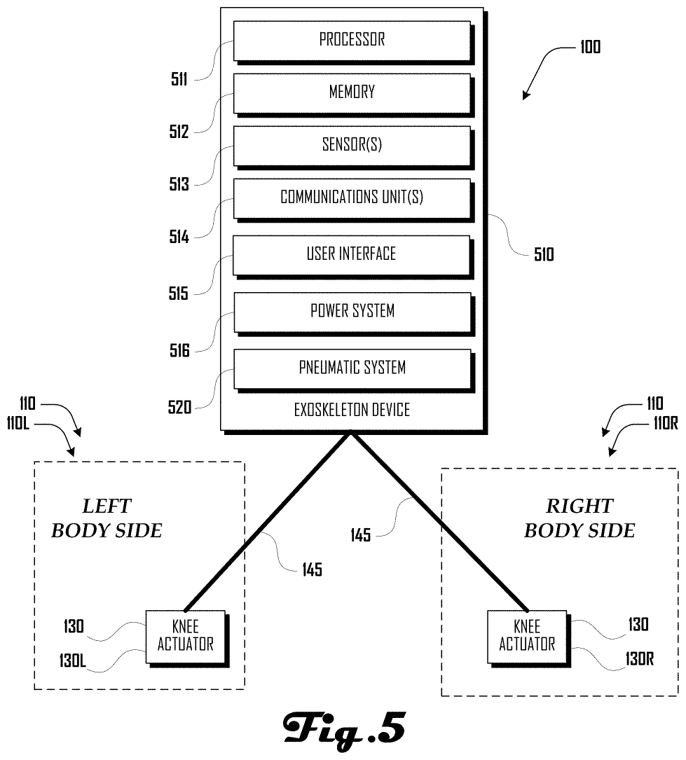

As shown in FIGS. 1-4, leg actuator units 110 can include an upper arm 115 and a lower arm 120 that are rotatably coupled via a joint 125. A bellows actuator 130 extends between the upper arm 115 and lower arm 120. One or more sets of cables 145 can be coupled to the bellows actuator 130 to introduce and/or remove fluid from the bellows actuator 130 to cause the bellows actuator 130 to expand and contract and to stiffen and soften, as discussed herein. As discussed herein, in various embodiments, such cables 145 can transmit power, communication signals, and the like to and/or from one or more bellows actuators 130. A backpack 155 can be worn by the user 101 and can hold various components of the exoskeleton system 100 such as a fluid source, control system, a power source, pneumatic system, and the like (see e.g., FIG. 5).

As shown in FIGS. 1-3, the leg actuator units 110L, 110R can be respectively coupled about the legs 102L, 102R of the user 101 with the joints 125 positioned at the knees 103L, 103R of the user 101 with the upper arms 115 of the leg actuator units 110L, 110R being coupled about the upper leg portions 104L, 104R of the user 101 via one or more couplers 150 (e.g., straps that surround the legs 102). The lower arms 120 of the leg actuator units 110L, 110R can be coupled about the lower leg portions 105L, 105R of the user 101 via one or more couplers 150.

The upper and lower arms 115, 120 of a leg actuator unit 110 can be coupled about the leg 102 of a user 101 in various suitable ways. For example, FIGS. 1-3 illustrate an example where the upper and lower arms 115, 120 and joint 125 of the leg actuator unit 110 are coupled along lateral faces (sides) of the top and bottom portions 104, 105 of the leg 102. As shown in the example of FIGS. 1-3, the upper arm 115 can be coupled to the upper leg portion 104 of a leg 102 above the knee 103 via two couplers 150 and the lower arm 120 can be coupled to the lower leg portion 105 of a leg 102 below the knee 103 via two couplers 150.

Specifically, the upper arm 115 can be coupled to the upper leg portion 104 of the leg 102 above the knee 103 via a first set of couplers 250A that includes a first and second coupler 150A, 150B. The first and second couplers 150A, 150B can be joined by a rigid plate assembly 215 disposed on a lateral side of the upper leg portion 104 of the leg 102, with straps 151 of the first and second couplers 150A, 150B extending around the upper leg portion 104 of the leg 102. The upper arm 115 can be coupled to the plate assembly 215 on a lateral side of the upper leg portion 104 of the leg 102, which can transfer force generated by the upper arm 115 to the upper leg portion 104 of the leg 102.

The lower arm 120 can be coupled to the lower leg portion 105 of a leg 102 below the knee 103 via a second set of couplers 250B that includes a third and fourth coupler 150C, 150D. A coupling branch unit 220 can extend from a distal end of, or be defined by a distal end of the lower arm 120. The coupling branch unit 220 can comprise a first branch 221 that extends from a lateral position on the lower leg portion 105 of the leg 102, curving upward and toward the anterior (front) of the lower leg portion 105 to a first attachment 222 on the anterior of the lower leg portion 105 below the knee 103, with the first attachment 222 joining the third coupler 150C and the first branch 221 of the coupling branch unit 220. The coupling branch unit 220 can comprise a second branch 223 that extends from a lateral position on the lower leg portion 105 of the leg 102, curving downward and toward the posterior (back) of the lower leg portion 105 to a second attachment 224 on the posterior of the lower leg portion 105 below the knee 103, with the second attachment 224 joining the fourth coupler 150D and the second branch 223 of the coupling branch unit 220.

As shown in the example of FIGS. 1-3, the fourth coupler 150D can be configured to surround and engage the boot 191 of a user. For example, the strap 151 of the fourth coupler 150D can be of a size that allows the fourth coupler 150D to surround the larger diameter of a boot 191 compared to the lower portion 105 of the leg 102 alone. Also, the length of the lower arm 120 and/or coupling branch unit 220 can be of a length sufficient for the fourth coupler 150D to be positioned over a boot 191 instead of being of a shorter length such that the fourth coupler 150D would surround a section of the lower portion 105 of the leg 102 above the boot 191 when the leg actuator unit 110 is worn by a user.

Attaching to the boot 191 can vary across various embodiments. In one embodiment, this attachment can be accomplished through a flexible strap that wraps around the circumference of boot 191 to affix the leg actuator unit 110 to the boot 191 with the desired amount of relative motion between the leg actuator unit 110 and the strap. Other embodiments can work to restrict various degrees of freedom while allowing the desired amount of relative motion between the leg actuator unit 110 and the boot 191 in other degrees of freedom. One such embodiment can include the use of a mechanical clip that connects to the back of the boot 191 that can provide a specific mechanical connection between the device and the boot 191. Various embodiments can include but are not limited to the designs listed previously, a mechanical bolted connection, a rigid strap, a magnetic connection, an electro-magnetic connection, an electromechanical connection, an insert into the user's boot, a rigid or flexible cable, or a connection directly to a boot.

Another aspect of the exoskeleton system 100 can be fit components used to secure the exoskeleton system 100 to the user 101. Since the function of the exoskeleton system 100 in various embodiments can rely heavily on the fit of the exoskeleton system 100 efficiently transmitting forces between the user 101 and the exoskeleton system 100 without the exoskeleton system 100 significantly drifting on the body 101 or creating discomfort, improving the fit of the exoskeleton system 100 and monitoring the fit of the exoskeleton system 100 to the user over time can be desirable for the overall function of the exoskeleton system 100 in some embodiments.

In various examples, different couplers 150 can be configured for different purposes, with some couplers 150 being primarily for the transmission of forces, with others being configured for secure attachment of the exoskeleton system 100 to the body 101. In one preferred embodiment for a single knee system, a coupler 150 that sits on the lower leg 105 of the user 101 (e.g., one or both of couplers 150C, 150D) can be intended to target body fit, and as a result, can remain flexible and compliant to conform to the body of the user 101. Alternatively, in this embodiment a coupler 150 that affixes to the front of the user's thigh on an upper portion 104 of the leg 102 (e.g., one or both of couplers 150A, 150B) can be intended to target power transmission needs and can have a stiffer attachment to the body than other couplers 150 (e.g., one or both of couplers 150C, 150D). Various embodiments can employ a variety of strapping or coupling configurations, and these embodiments can extend to include any variety of suitable straps, couplings, or the like, where two parallel sets of coupling configurations are meant to fill these different needs.

In some cases, the design of the joint 125 can improve the fit of the exoskeleton system 100 on the user. In one embodiment, the joint 125 of a single knee leg actuator unit 110 can be designed to use a single pivot joint that has some deviations with the physiology of the knee joint. Another embodiment uses a polycentric knee joint to better fit the motion of the human knee joint, which in some examples can be desirably paired with a very well fit leg actuator unit 110. Various embodiments of a joint 125 can include but are not limited to the example elements listed above, a ball and socket joint, a four-bar linkage, and the like.

Some embodiments can include fit adjustments for anatomical variations in varus or valgus angles in the lower leg 105. One preferred embodiment includes an adjustment incorporated into a leg actuator unit 110 in the form of a cross strap that spans the joint of the knee 103 of the user 101, which can be tightened to provide a moment across the knee joint in the frontal plane which varies the nominal resting angle. Various embodiments can include but are not limited to the following: a strap that spans the joint 125 to vary the operating angle of the joint 125; a mechanical assembly including a screw that can be adjusted to vary the angle of the joint 125; mechanical inserts that can be added to the leg actuator unit 110 to discreetly change the default angle of the joint 125 for the user 101, and the like.

In various embodiments, the leg actuator unit 110 can be configured to remain suspended vertically on the leg 102 and remain appropriately positioned with the joint of the knee 103. In one embodiment, a coupler 150 associated with a boot 191 (e.g., coupler 150D) can provide a vertical retention force for a leg actuator unit 110. Another embodiment uses a coupler 150 positioned on the lower leg 105 of the user 101 (e.g., one or both of couplers 150C, 150D) that exerts a vertical force on the leg actuator unit 110 by reacting on the calf of the user 101. Various embodiments can include but are not limited to the following: suspension forces transmitted through a coupler 150 on the boot (e.g., coupler 150D) or another embodiment of the boot attachment discussed previously; suspension forces transmitted through an electronic and/or fluidic cable assembly; suspension forces transmitted through a connection to a waist belt; suspension forces transmitted through a mechanical connection to a backpack 155 or other housing for the exoskeleton device 510 and/or pneumatic system 520 (see FIG. 5); suspension forces transmitted through straps or a harness to the shoulders of the user 101, and the like.

In various embodiments, a leg actuator unit 110 can be spaced apart from the leg 102 of the user with a limited number of attachments to the leg 102. For example, in some embodiments, the leg actuator unit 110 can consist or consist essentially of three attachments to the leg 102 of the user 101, namely via the first and second attachments 222, 224 and 215. In various embodiments, the couplings of the leg actuator unit 110 to the lower leg portion 105 can consist or consist essentially of a first and second attachment on the anterior and posterior of the lower leg portion 105. In various embodiments, the coupling of the leg actuator unit 110 to the upper leg portion 104 can consist or consist essentially of a single lateral coupling, which can be associated with one or more couplers 150 (e.g., two couplers 150A, 150B as shown in FIGS. 1-4). In various embodiments, such a configuration can be desirable based on the specific force-transfer for use during a subject activity. Accordingly, the number and positions of attachments or coupling to the leg 102 of the user 101 in various embodiments is not a simple design choice and can be specifically selected for one or more selected target user activities.

While specific embodiments of couplers 150 are illustrated herein, in further embodiments, such components discussed herein can be operably replaced by an alternative structure to produce the same functionality. For example, while straps, buckles, padding and the like are shown in various examples, further embodiments can include couplers 150 of various suitable types and with various suitable elements. For example, some embodiments can include Velcro hook-and-loop straps, or the like.

FIGS. 1-3 illustrate an example of an exoskeleton system 100 where the joint 125 is disposed laterally and adjacent to the knee 103 with a rotational axis of the joint 125 being disposed parallel to a rotational axis of the knee 103. In some embodiments, the rotational axis of the joint 125 can be coincident with the rotational axis of the knee 103. In some embodiments, a joint can be disposed on the anterior of the knee 103, posterior of the knee 103, inside of the knee 103, or the like.

In various embodiments, the joint structure 125 can constrain the bellows actuator 130 such that force created by actuator fluid pressure within the bellows actuator 130 can be directed about an instantaneous center (which may or may not be fixed in space). In some cases of a revolute or rotary joint, or a body sliding on a curved surface, this instantaneous center can coincide with the instantaneous center of rotation of the joint 125 or a curved surface. Forces created by a leg actuator unit 110 about a rotary joint 125 can be used to apply a moment about an instantaneous center as well as still be used to apply a directed force. In some cases of a prismatic or linear joint (e.g., a slide on a rail, or the like), the instantaneous center can be kinematically considered to be located at infinity, in which case the force directed about this infinite instantaneous center can be considered as a force directed along the axis of motion of the prismatic joint. In various embodiments, it can be sufficient for a rotary joint 125 to be constructed from a mechanical pivot mechanism. In such an embodiment, the joint 125 can have a fixed center of rotation that can be easy to define, and the bellows actuator 130 can move relative to the joint 125. In a further embodiment, it can be beneficial for the joint 125 to comprise a complex linkage that does not have a single fixed center of rotation. In yet another embodiment, the joint 125 can comprise a flexure design that does not have a fixed joint pivot. In still further embodiments, the joint 125 can comprise a structure, such as a human joint, robotic joint, or the like.

In various embodiments, leg actuator unit 110 (e.g., comprising bellows actuator 130, joint structure 125, and the like) can be integrated into a system to use the generated directed force of the leg actuator unit 110 to accomplish various tasks. In some examples, a leg actuator unit 110 can have one or more unique benefits when the leg actuator unit 110 is configured to assist the human body or is included into a powered exoskeleton system 100. In an example embodiment, the leg actuator unit 110 can be configured to assist the motion of a human user about the user's knee joint 103. To do so, in some examples, the instantaneous center of the leg actuator unit 110 can be designed to coincide or nearly coincide with the instantaneous center of rotation of the knee 103 of a user 101. In one example configuration, the leg actuator unit 110 can be positioned lateral to the knee joint 103 as shown in FIGS. 1-3. In various examples, the human knee joint 103 can function as (e.g., in addition to or in place of) the joint 125 of the leg actuator unit 110.

For clarity, example embodiments discussed herein should not be viewed as a limitation of the potential applications of the leg actuator unit 110 described within this disclosure. The leg actuator unit 110 can be used on other joints of the body including but not limited to one or more elbow, one or more hip, one or more finger, one or more ankle, spine, or neck. In some embodiments, the leg actuator unit 110 can be used in applications that are not on the human body such as in robotics, for general purpose actuation, animal exoskeletons, or the like.

Also, embodiments can be used for or adapted for various suitable applications such as tactical, medical, or labor applications, and the like. Examples of such applications can be found in U.S. patent application Ser. No. 15/823,523, filed Nov. 27, 2017, entitled “PNEUMATIC EXOMUSCLE SYSTEM AND METHOD” with attorney docket number 0110496-002US1 and U.S. patent application Ser. No. 15/953,296, filed Apr. 13, 2018, entitled “LEG EXOSKELETON SYSTEM AND METHOD” with attorney docket number 0110496-004US0, which are incorporated herein by reference.

Some embodiments can apply a configuration of a leg actuator unit 110 as described herein for linear actuation applications. In an example embodiment, the bellows actuator 130 can comprise a two-layer impermeable/inextensible construction, and one end of one or more constraining ribs can be fixed to the bellows actuator 130 at predetermined positions. The joint structure 125 in various embodiments can be configured as a series of slides on a pair of linear guide rails, where the remaining end of one or more constraining ribs is connected to a slide. The motion and force of the fluidic actuator can therefore be constrained and directed along the linear rail.

FIG. 5 is a block diagram of an example embodiment of an exoskeleton system 100 that includes an exoskeleton device 510. While a pneumatic system 520 is used in the example of FIG. 5, further embodiments can include any suitable fluidic system or a pneumatic system 520 can be absent in some embodiments, such as where an exoskeleton system 100 is actuated by electric motors, or the like.

The exoskeleton device 510 in this example comprises a processor 511, a memory 512, one or more sensors 513 a communication unit 514, a user interface 515, a power source 516 and a pneumatic system 520. In various embodiments, fluid (e.g., air), electrical power, communication signals, and the like can be communicated to and/or from the actuator units 110 via respective cables 145. For example, the cables 145 can be configured to convey air from a fluid source (e.g., of the pneumatic system 520) to the actuators 130, which can cause actuation of the actuators 130 as discussed herein. In various embodiments, the cables 145 can be configured to provide air to the actuators 130 separately such that the actuators 130 can be selectively controlled separately.

Additionally, in various embodiments, the lines can be configured to transmit electrical power from the power system 516 (e.g., from a battery) to the actuator units 110, which can be used at the actuator units 110 to power elements of the actuator units 110 such as pneumatic valves, sensors, an embedded system, an interface, a computing system, and the like. In various embodiments, the actuator units 110 and exoskeleton device 510 can be configured to communicate via the cables 145. For example, in various embodiments, the exoskeleton device 510 can communicate control signals (e.g., via the communication unit(s) 514) to the actuator units 110, which can be configured to control actuation of the actuator units 110, output of an interface, or the like. In further embodiments, any suitable communications or data can be sent to the actuator units 110 and/or actuators 130 via the cables 145, which can be via any suitable communication protocol. Also, in various embodiments, communications or data can be sent to the exoskeleton device 510 from the actuator units 110 and/or actuators 130 via the cables 145. For example, sensor data, status data, configuration data, pneumatic data, or the like, can be sent to the exoskeleton device 510 from the actuator units 110 and/or actuators 130 via the cables 145.

In accordance with some embodiments, communication to or from or between the exoskeleton device 510 and the actuator units 110 and/or actuators 130 can comprise wireless communication in addition to or alternative to communication via the cables 145. However, in some embodiments, communications to or from or between the exoskeleton device 510 and the actuator units 110 and/or actuators 130 can be exclusively via the cables 145, with the system being incapable of wireless communications to or from or between the exoskeleton device 510 and the actuator units 110 and/or actuators 130.

Also, as discussed in more detail herein, in various embodiments, the cables 145 can be configured as a unitary structure capable of transmitting electrical power, fluid (e.g., air), and/or communications to, from or between the exoskeleton device 510 and the actuator units 110 and/or actuators 130. In other words, various embodiments, can have, consist of or consist essentially of only a single unitary cable 145 for transmitting electrical power, fluid (e.g., air), and/or communications to, from or between the exoskeleton device 510 and respective actuator units 110 and/or respective actuators 130 via one or more electrical power lines (e.g., wires), one or more fluid lines (e.g., tubes), one or more communication lines (e.g., wires, fiberoptic, etc.), and the like.

It can be desirable in some examples for the cable(s) 145 to be strong to hold up against unintentional strain. In a preferred embodiment one or more electrical power lines, one or more fluid lines, and/or one or more communication lines are unified into one cable 145. In such an embodiment the one or more electrical power lines, one or more fluid lines, and/or one or more communication lines can run in parallel and can be encased in a sheath individually and/or collectively (e.g., with a medical grade material). For example, encasing such lines to define a cable 145 can include various insulation, inner/outer sheaths, and the like. Encasing the one or more electrical power lines, one or more fluid lines, and/or one or more communication lines together with a strong material in some embodiments can help protect them from environmental factors, such as water, snow, or sand. In another embodiment, the one or more electrical power lines, one or more fluid lines, and/or one or more communication lines may run in parallel together and are attached together in various suitable ways (e.g., by zip ties, tape or adhesives). Whether the one or more electrical power lines, one or more fluid lines, and/or one or more communication lines are one component or more, by attaching them together, in various embodiments weaker electronic wires may no longer need to hold the high strain that stronger fluidic tubes can withstand.

It can also be desirable to reduce the length of cables 145 hanging outside of the pack, which can snag onto other objects. One preferred set of embodiments includes retractable cables 145. In at least some of such embodiments, it can be preferable for the retractable cables 145 to be accomplished inside a backpack 155, with the cables 145 configured to have a small mechanical retention force to maintain cables 145 that are pulled tight against the user with reduced slack remaining in the cable(s) 145. This can be done in some embodiments with a linear spring attached to the cables or a rotating spool with a rotational spring, both of which may pull the cable back into the power pack (e.g., backpack 155) in various examples. Further embodiments can be used to organize or route the cables 145 so that they do not snag, such as integrating them into the user's clothing, or clipping onto other sections of the power pack with hooks, straps, buttons or magnets.

Another aspect of the cable(s) 145 can be mounting to the backpack 155, actuator unit 110 and/or actuator 130. In a preferred embodiment, pigtail type connections are used. In various pigtail type connections, the cable 145 extends through a rigid housing of a given device and a portion of the cable connector 600 is at the end of the cable 145. Specifically, these connections in some examples can utilize inline connections as opposed to panel-mount connections. This can reduce the shear stress on the internal electronics and mechanical connection, if, for example a cable 145 is accidentally snagged by an object. Various other types of line mounts can be used including, but not limited to, panel-mounted connections.

The plurality of actuators 130 include a pair of knee-actuators 130 Land 13OR that are positioned on the right and left side of a body 100. For example, as discussed above, the example exoskeleton system 100 shown in FIG. 5 can comprise a left and right leg actuator unit 110L, 110R on respective sides of the body 101 as shown in FIGS. 1 and 2 with one or both of the exoskeleton device 510 and pneumatic system 520, or one or more components thereof, stored within or about a backpack 155 (see FIG. 1) or otherwise mounted, worn or held by a user 101.

Accordingly, in various embodiments, the exoskeleton system 100 can be a completely mobile and self-contained system that is configured to be powered and operated for an extended period of time without an external power source during various user activities. The size, weight and configuration of the actuator unit(s) 110, exoskeleton device 510 and pneumatic system 520 can therefore be configured in various embodiments for such mobile and self-contained operation.

In various embodiments, the example system 100 can be configured to move and/or enhance movement of the user 101 wearing the exoskeleton system 100. For example, the exoskeleton device 510 can provide instructions to the pneumatic system 520, actuator units 110 and/or actuators 130, which can selectively inflate and/or deflate the bellows actuators 130 via the cables 145. For example, fluid can be sent to the actuator units 110 and/or actuators 130 via the cables 145 with control of such fluid being via fluid valves or other suitable elements at the exoskeleton device 510, actuator units 110 and/or actuators 130. Such selective inflation and/or deflation of the bellows actuators 130 can move and/or support one or both legs 102 to generate and/or augment body motions such as walking, running, jumping, climbing, lifting, throwing, squatting, skiing or the like.

In some cases, the exoskeleton system 100 can be designed to support multiple configurations in a modular configuration. For example, one embodiment is a modular configuration that is designed to operate in either a single knee configuration or in a double knee configuration as a function of how many of the actuator units 110 are donned by the user 101. For example, the exoskeleton device 510 can determine how many actuator units 110 are coupled to the pneumatic system 520 and/or exoskeleton device 510 (e.g., one or two actuator units 110) and the exoskeleton device 510 can change operating capabilities based on the number of actuator units 110 detected.

In further embodiments, the pneumatic system 520 can be manually controlled, configured to apply a constant pressure, or operated in any other suitable manner. In some embodiments, such movements can be controlled and/or programmed by the user 101 that is wearing the exoskeleton system 100 or by another person. In some embodiments, the exoskeleton system 100 can be controlled by movement of the user 101. For example, the exoskeleton device 510 can sense that the user is walking and carrying a load and can provide a powered assist to the user via the actuators 130 to reduce the exertion associated with the load and walking. Similarly, where a user 101 wears the exoskeleton system 100, the exoskeleton system 100 can sense movements of the user 101 and can provide a powered assist to the user via the actuators 130 to enhance or provide an assist to the user while skiing.

Accordingly, in various embodiments, the exoskeleton system 130 can react automatically without direct user interaction. In further embodiments, movements can be controlled in real-time by user interface 515 such as a controller, joystick, voice control or thought control. Additionally, some movements can be pre-preprogrammed and selectively triggered (e.g., walk forward, sit, crouch) instead of being completely controlled. In some embodiments, movements can be controlled by generalized instructions (e.g., walk from point A to point B, pick up box from shelf A and move to shelf B).

The user interface 515 can allow the user 101 to control various aspects of the exoskeleton system 100 including powering the exoskeleton system 100 on and off; controlling movements of the exoskeleton system 100; configuring settings of the exoskeleton system 100, and the like. The user interface 515 can include various suitable input elements such as a touch screen, one or more buttons, audio input, and the like. The user interface 515 can be located in various suitable locations about the exoskeleton system 100. For example, in one embodiment, the user interface 515 can be disposed on a strap of a backpack 155, or the like. In some embodiments, the user interface can be defined by a user device such as smartphone, smartwatch, wearable device, or the like.

In various embodiments, the power source 516 can be a mobile power source that provides the operational power for the exoskeleton system 100. In one preferred embodiment, the power pack unit contains some or all of the pneumatic system 520 (e.g., a compressor) and/or power source (e.g., batteries) required for the continued operation of pneumatic actuation of the leg actuator units 110. The contents of such a power pack unit can be correlated to the specific actuation approach configured to be used in the specific embodiment. In some embodiments, the power pack unit will only contain batteries which can be the case in an electromechanically actuated system or a system where the pneumatic system 520 and power source 516 are separate. Various embodiments of a power pack unit can include but are not limited to a combination of one or more of the following items: pneumatic compressor, batteries, stored high-pressure pneumatic chamber, hydraulic pump, pneumatic safety components, electric motor, electric motor drivers, microprocessor, and the like. Accordingly, various embodiments of a power pack unit can include one or more of elements of the exoskeleton device 510 and/or pneumatic system 520.

Such components can be configured on the body of a user 101 in a variety of suitable ways. One preferred embodiment is the inclusion of a power pack unit in a torso-worn pack that is not operably coupled to the leg actuator units 110 in any manner that transmits substantial mechanical forces to the leg actuator units 110. Another embodiment includes the integration of the power pack unit, or components thereof, into the leg actuator units 110 themselves. Various embodiments can include but are not limited to the following configurations: torso-mounted in a backpack, torso-mounted in a messenger bag, hip-mounted bag, mounted to the leg, integrated into the brace component, and the like. Further embodiments can separate the components of the power pack unit and disperse them into various configurations on the user 101. Such an embodiment may configure a pneumatic compressor on the torso of the user 101 and then integrate the batteries into the leg actuator units 110 of the exoskeleton system 100.

One aspect of the power supply 516 in various embodiments is that it must be connected to the brace component in such a manner as to pass the operable system power to the brace for operation. One preferred embodiment is the use of electrical cables (e.g., as part of unified cable 145) to connect the power supply 516 and the leg actuator units 110. Other embodiments can use electrical cables separate from cables 145, wireless power transmission, and/or local batteries to deliver electrical power. Various embodiments can include but are not limited to any configuration of the following connections, which may or may not be part of a unified cable 145: pneumatic hosing, hydraulic hosing, electrical cables, wireless communication, wireless power transfer, and the like.

In some embodiments, it can be desirable to include secondary features that extend the capabilities of a cable connection (e.g., cables 145) between the leg actuator units 110 and elements of the exoskeleton device 510 such as the power supply 516 and/or pneumatic system 520. One preferred embodiment includes retractable cables that are configured to have a small mechanical retention force to maintain cables 145 that are pulled tight against the user with reduced slack remaining in the cables 145. Various embodiments can include, but are not limited to a combination of the following secondary features: retractable cables, a single cable 145 including both fluidic and electrical power, magnetically connected electrical cables, mechanical quick releases, breakaway connections designed to release at a specified pull force, integration into mechanical retention features on the user's clothing, a unified singular cable 145 for power, air and/or communications, and the like. Yet another embodiment can include routing the cables 145 in such a way as to minimize geometric differences between the user 101 and lengths of the cables 145. One such embodiment in a dual knee configuration with a torso power supply can be routing the cables 145 along the user's lower torso to connect the right side of a power supply bag with the left knee of the user. Such a routing can allow the geometric differences in length throughout the user's normal range of motion.

One specific additional feature that can be a concern in some embodiments is the need for proper heat management of the exoskeleton system 100. As a result, there are a variety of features that can be integrated specifically for the benefit of controlling heat. One preferred embodiment integrates exposed heat sinks to the environment that allow elements of the exoskeleton device 510 and/or pneumatic system 520 to dispel heat directly to the environment through unforced cooling using ambient airflow. Another embodiment directs the ambient air through internal air channels in a backpack 155 or other housing to allow for internal cooling. Yet another embodiment can extend upon this capability by introducing scoops on a backpack 155 or other housing in an effort to allow air flow through the internal channels. Various embodiments can include but are not limited to the following: exposed heat sinks that are directly connected to a high heat component; a water-cooled or fluid-cooled heat management system; forced air cooling through the introduction of a powered fan or blower; external shielded heat sinks to protect them from direct contact by a user, and the like.

In some cases, it may be beneficial to integrate additional features into the structure of the backpack 155 or other housing to provide additional features to the exoskeleton system 100. One preferred embodiment is the integration of mechanical attachments to support storage of the leg actuator units 110 along with the exoskeleton device 510 and/or pneumatic system 520 in a small package. Such an embodiment can include a deployable pouch that can secure the leg actuator units 110 against the backpack 155 along with mechanical clasps that hold the upper or lower arms 115, 120 of the actuator units 110 to the backpack 155. Another embodiment is the inclusion of storage capacity into the backpack 155 so the user 101 can hold additional items such as a water bottle, food, personal electronics, and other personal items. Various embodiments can include but are not limited to other additional features such as the following: a warming pocket which is heated by hot airflow from the exoskeleton device 510 and/or pneumatic system 520; air scoops to encourage additional airflow internal to the backpack 155; strapping to provide a closer fit of the backpack 155 on the user, waterproof storage, temperature-regulated storage, and the like.

In a modular configuration, it may be required in some embodiments that the exoskeleton device 510 and/or pneumatic system 520 can be configured to support the electrical power, fluidic power, sensing and control requirements and capabilities of various potential configurations of the exoskeleton system. One preferred embodiment can include an exoskeleton device 510 and/or pneumatic system 520 that can be tasked with powering a dual knee configuration or a single knee configuration (i.e., with one or two leg actuator units 110 on the user 101). Such an exoskeleton system 100 can support the requirements of both configurations and then appropriately configure electrical power, fluidic power, sensing and control based on a determination or indication of a desired operating configuration. Various embodiments exist to support an array of potential modular system configurations, such as multiple batteries, and the like.

In various embodiments, the exoskeleton system 100 can be operable to perform methods or portions of methods described in more detail below or in related applications incorporated herein by reference. For example, the memory 512 can include non-transitory computer readable instructions (e.g., software), which if executed by the processor 511, can cause the exoskeleton system 100 to perform methods or portions of methods described herein or in related applications incorporated herein by reference.

This software can embody various methods that interpret signals from the sensors 513 or other sources to determine how to best operate the exoskeleton system 100 to provide the desired benefit to the user. The specific embodiments described below should not be used to imply a limit on the sensors 513 that can be applied to such an exoskeleton system 100 or the source of sensor data. While some example embodiments can require specific information to guide decisions, it does not create an explicit set of sensors 513 that an exoskeleton system 100 will require and further embodiments can include various suitable sets of sensors 513. Additionally, sensors 513 can be located at various suitable locations on an exoskeleton system 100 including as part of an exoskeleton device 510, pneumatic system 520, one or more fluidic actuator 130, or the like. Accordingly, the example illustration of FIG. 5 should not be construed to imply that sensors 513 are exclusively disposed at or part of an exoskeleton device 510 and such an illustration is merely provided for purposes of simplicity and clarity.

One aspect of control software can be the operational control of leg actuator units 110, exoskeleton device 510 and pneumatic system 520 to provide the desired response. There can be various suitable responsibilities of the operational control software. For example, as discussed in more detail below, one can be low-level control which can be responsible for developing baseline feedback for operation of the leg actuator units 110, exoskeleton device 510 and pneumatic system 520. Another can be intent recognition which can be responsible for identifying the intended maneuvers of the user 101 based on data from the sensors 513 and causing the exoskeleton system 100 to operate based on one or more identified intended maneuvers. A further example can include reference generation, which can include selecting the desired torques the exoskeleton system 100 should generate to best assist the user 101. It should be noted that this example architecture for delineating the responsibilities of the operational control software is merely for descriptive purposes and in no way limits the wide variety of software approaches that can be deployed on further embodiments of an exoskeleton system 100.

One method implemented by control software can be for the low-level control and communication of the exoskeleton system 100. This can be accomplished via a variety of methods as required by the specific joint and need of the user. In a preferred embodiment, the operational control is configured to provide a desired torque by the leg actuator unit 110 at the user's joint. In such a case, the exoskeleton system 100 can create low-level feedback to achieve a desired joint torque by the leg actuator units 110 as a function of feedback from the sensors 513 of the exoskeleton system 100. For example, such a method can include obtaining sensor data from one or more sensors 513, determining whether a change in torque by the leg actuator unit 110 is necessary, and if so, causing the pneumatic system 520 to change the fluid state of the leg actuator unit 110 to achieve a target joint torque by the leg actuator unit 110. Various embodiments can include, but are not limited to, the following: current feedback; recorded behavior playback; position-based feedback; velocity-based feedback; feedforward responses; volume feedback which controls a fluidic system 520 to inject a desired volume of fluid into an actuator 130, and the like.

Another method implemented by operational control software can be for intent recognition of the user's intended behaviors. This portion of the operational control software, in some embodiments, can indicate any array of allowable behaviors that the system 100 is configured to account for. In one preferred embodiment, the operational control software is configured to identify two specific states: Walking, and Not Walking. In such an embodiment, to complete intent recognition, the exoskeleton system 100 can use user input and/or sensor readings to identify when it is safe, desirable or appropriate to provide assistive actions for walking. For example, in some embodiments, intent recognition can be based on input received via the user interface 515, which can include an input for Walking, and Not Walking. Accordingly, in some examples, the use interface can be configured for a binary input consisting of Walking, and Not Walking.

In some embodiments, a method of intent recognition can include the exoskeleton device 510 obtaining data from the sensors 513 and determining, based at least in part of the obtained data, whether the data corresponds to a user state of Walking, and Not Walking. Where a change in state has been identified, the exoskeleton system 100 can be re-configured to operate in the current state. For example, the exoskeleton device 510 can determine that the user 101 is in a Not Walking state such as sitting and can configure the exoskeleton system 100 to operate in a Not Walking configuration. For example, such a Not Walking configuration can, compared to a Walking configuration, provide for a wider range of motion; provide no torque or minimal torque to the leg actuation units 110; save power and fluid by minimizing processing and fluidic operations; cause the system to be alert for supporting a wider variety of non-skiing motion, and the like.

The exoskeleton device 510 can monitor the activity of the user 101 and can determine that the user is walking or is about to walk (e.g., based on sensor data and/or user input), and can then configure the exoskeleton system 100 to operate in a Walking configuration. For example, such a Walking configuration, compared to a Not Walking configuration, can allow for a more limited range of motion that would be present during skiing (as opposed to motions during non-walking); provide for high or maximum performance by increasing the processing and fluidic response of the exoskeleton system 100 to support skiing; and the like. When the user 101 finishes a walking session, is identified as resting, or the like, the exoskeleton system 100 can determine that the user is no longer walking (e.g., based on sensor data and/or user input) and can then configure the exoskeleton system 100 to operate in the Not Walking configuration.

In some embodiments, there can be a plurality of Walking states, or Walking sub-states that can be determined by the exoskeleton system 100, including hard walking, moderate walking, light walking, downhill, uphill, jumping, recreational, sport, running, and the like (e.g., based on sensor data and/or user input). Such states can be based on the difficulty of the walking, ability of the user, terrain, weather conditions, elevation, angle of the walking surface, desired performance level, power-saving, and the like. Accordingly, in various embodiments, the exoskeleton system 100 can adapt for various specific types of walking or movement based on a wide variety of factors.

Another method implemented by operational control software can be the development of desired referenced behaviors for the specific joints providing assistance. This portion of the control software can tie together identified maneuvers with the level control. For example, when the exoskeleton system 100 identifies an intended user maneuver, the software can generate reference behaviors that define the torques, or positions desired by the actuators 130 in the leg actuation units 110. In one embodiment, the operational control software generates references to make the leg actuation units 110 simulate a mechanical spring at the knee 103 via the configuration actuator 130. The operational control software can generate torque references at the knee joints that are a linear function of the knee joint angle. In another embodiment, the operational control software generates a volume reference to provide a constant standard volume of air into a pneumatic actuator 130. This can allow the pneumatic actuator 130 to operate like a mechanical spring by maintaining the constant volume of air in the actuator 130 regardless of the knee angle, which can be identified through feedback from one or more sensors 513.

In another embodiment, a method implemented by the operational control software can include evaluating the balance of the user 101 while walking, moving, standing, or running and directing torque in such a way to encourage the user 101 to remain balanced by directing knee assistance to the leg 102 that is on the outside of the user's current balance profile. Accordingly, a method of operating an exoskeleton system 100 can include the exoskeleton device 510 obtaining sensor data from the sensors 510 indicating a balance profile of a user 101 based on the configuration of left and right leg actuation units 110L, 110R and/or environmental sensors such as position sensors, accelerometers, and the like. The method can further include determining a balance profile based on the obtained data, including an outside and inside leg, and then increasing torque to the actuation unit 110 associated with the leg 102 identified as the outside leg.

Various embodiments can use but are not limited to kinematic estimates of posture, joint kinetic profile estimates, as well as observed estimates of body pose. Various other embodiments exist for methods of coordinating two legs 102 to generate torques including but not limited to guiding torque to the most bent leg; guiding torque based on the mean amount of knee angle across both legs; scaling the torque as a function of speed or acceleration; and the like. Yet another embodiment can include a combination of various individual reference generation methods in a variety of matters which include but are not limited to a linear combination, a maneuver specific combination, or a non-linear combination.

In another embodiment, an operational control method can blend two primary reference generation techniques: one reference focused on static assistance and one reference focused on leading the user 101 into their upcoming behavior. In some examples, the user 101 can select how much predictive assistance is desired while using the exoskeleton system 100. For example, by a user 101 indicating a large amount of predictive assistance, the exoskeleton system 100 can be configured to be very responsive and may be well configured for a skilled operator on a challenging terrain. The user 101 could also indicate a desire for a very low amount of predictive assistance, which can result in slower system performance, which may be better tailored towards a learning user or less challenging terrain.

Various embodiments can incorporate user intent in a variety of manners and the example embodiments presented above should not be interpreted as limiting in any way. For example, method of determining and operating an exoskeleton system 100 can include systems and method of U.S. patent application Ser. No. 15/887,866, filed Feb. 2, 2018, entitled “SYSTEM AND METHOD FOR USER INTENT RECOGNITION,” having attorney docket number 0110496-003US0, which is incorporated herein by reference. Also, various embodiments can use user intent in a variety of manners including as a continuous unit, or as a discrete setting with only a few indicated values.

At times it can be beneficial for operational control software to manipulate its control to account for a secondary or additional objective in order to maximize device performance or user experience. In one embodiment, the exoskeleton system 100 can provide an elevation-aware control over a central compressor or other components of a pneumatic system 520 to account for the changing density of air at different elevations. For example, operational control software can identify that the system is operating at a higher elevation based on data from sensors 513, or the like, and provide more current to the compressor in order to maintain electrical power consumed by the compressor. Accordingly, a method of operating a pneumatic exoskeleton system 100 can include obtaining data indicating air density where the pneumatic exoskeleton system 100 is operating (e.g., elevation data), determining optimal operating parameters of the pneumatic system 520 based on the obtained data, and configuring operation based on the determined optimal operating parameters. In further embodiments, operation of a pneumatic exoskeleton system 100 such as operating volumes can be tuned based on environmental temperature, which may affect air volumes.

In another embodiment, the exoskeleton system 100 can monitor the ambient audible noise levels and vary the control behavior of the exoskeleton system 100 to reduce the noise profile of the system. For example, when a user 101 is in a quiet public place or quietly enjoying a location alone or with others, noise associated with actuation of the leg actuation units 110 can be undesirable (e.g., noise of running a compressor or inflating or deflating actuators 130). Accordingly, in some embodiments, the sensors 513 can include a microphone that detects ambient noise levels and can configure the exoskeleton system 100 to operate in a quiet mode when ambient noise volume is below a certain threshold. Such a quiet mode can configure elements of a pneumatic system 520 or actuators 130 to operate more quietly, or can delay or reduce frequency of noise made by such elements.

In the case of a modular system, it can be desirable in various embodiments for operational control software to operate differently based on the number of leg actuation units 110 operational within the exoskeleton system 100. For example, in some embodiments, a modular dual-knee exoskeleton system 100 (see e.g., FIGS. 1 and 2) can also operate in a single-knee configuration where only one of two leg actuation units 110 are being worn by a user 101 (see e.g., FIGS. 3 and 4) and the exoskeleton system 100 can generate references differently when in a two-leg configuration compared to a single-leg configuration. Such an embodiment can use a coordinated control approach to generate references where the exoskeleton system 100 is using inputs from both leg actuation units 110 to determine the desired operation. However, in a single-leg configuration, the available sensor information may have changed, so in various embodiments the exoskeleton system 100 can implement a different control method. In various embodiments this can be done to maximize the performance of the exoskeleton system 100 for the given configuration or account for differences in available sensor information based on there being one or two leg actuation units 110 operating in the exoskeleton system 100.

Accordingly, a method of operating an exoskeleton system 100 can include a startup sequence where a determination is made by the exoskeleton device 510 whether one or two leg actuation units 110 are operating in the exoskeleton system 100; determining a control method based on the number of actuation units 110 that are operating in the exoskeleton system 100; and implementing and operating the exoskeleton system 100 with the selected control method. A further method operating an exoskeleton system 100 can include monitoring by the exoskeleton device 510 of actuation units 110 that are operating in the exoskeleton system 100, determining a change in the number of actuation units 110 operating in the exoskeleton system 100, and then determining and changing the control method based on the new number of actuation units 110 that are operating in the exoskeleton system 100.

For example, the exoskeleton system 100 can be operating with two actuation units 110 and with a first control method. The user 101 can disengage one of the actuation units 110, and the exoskeleton device 510 can identify the loss of one of the actuation units 110 and the exoskeleton device 510 can determine and implement a new second control method to accommodate loss of one of the actuation units 110. In some examples, adapting to the number of active actuation units 110 can be beneficial where one of the actuation units 110 is damaged or disconnected during use and the exoskeleton system 100 is able to adapt automatically so the user 101 can still continue working or moving uninterrupted despite the exoskeleton system 100 only having a single active actuation unit 110.

In various embodiments, operational control software can adapt a control method where user needs are different between individual actuation units 110 or legs 102. In such an embodiment, it can be beneficial for the exoskeleton system 100 to change the torque references generated in each actuation unit 110 to tailor the experience for the user 101. One example is of a dual knee exoskeleton system 100 (see e.g., FIG. 1) where a user 101 has significant weakness issues in a single leg 102, but only minor weakness issues in the other leg 102. In this example, the exoskeleton system 100 can be configured to scale down the output torques on the less-affected limb compared to the more-affected limb to best meet the needs of the user 101.

Such a configuration based on differential limb strength can be done automatically by the exoskeleton system 100 and/or can be configured via a user interface 516, or the like. For example, in some embodiments, the user 101 can perform a calibration test while using the exoskeleton system 100, which can test relative strength or weakness in the legs 102 of the user 101 and configure the exoskeleton system 100 based on identified strength or weakness in the legs 102. Such a test can identify general strength or weakness of legs 102 or can identify strength or weakness of specific muscles or muscle groups such as the quadriceps, calves, hamstrings, gluteus, gastrocnemius; femoris, sartorius, soleus, and the like.

Another aspect of a method for operating an exoskeleton system 100 can include control software that monitors the exoskeleton system 100. A monitoring aspect of such software can, in some examples, focus on monitoring the state of the exoskeleton system 100 and the user 101 throughout normal operation in an effort to provide the exoskeleton system 100 with situational awareness and understanding of sensor information in order to drive user understanding and device performance. One aspect of such monitoring software can be to monitor the state of the exoskeleton system 100 in order to provide device understanding to achieve a desired performance capability. A portion of this can be the development of a system body pose estimate. In one embodiment, the exoskeleton device 510 uses the onboard sensors 513 to develop a real-time understanding of the user's pose. In other words, data from sensors 513 can be used to determine the configuration of the actuation units 110, which along with other sensor data can in turn be used to infer a user pose or body configuration estimate of the user 101 wearing the actuation units 110.

At times, and in some embodiments, it can be unrealistic or impossible for the exoskeleton system 100 to directly sense all important aspects of the system pose due to the sensing modalities not existing or their inability to be practically integrated into the hardware. As a result, the exoskeleton system 100 in some examples can rely on a fused understanding of the sensor information around an underlying model of the user's body and the exoskeleton system 100 the user is wearing. In one embodiment of a dual leg knee assistance exoskeleton system 100, the exoskeleton device 510 can use an underlying model of the user's lower extremity and torso body segments to enforce a relational constraint between the otherwise disconnected sensors 513. Such a model can allow the exoskeleton system 100 to understand the constrained motion of the two legs 102 in that they are mechanically connected through the user's kinematic chain created by the body. This approach can be used to ensure that the estimates for knee orientation are properly constrained and biomechanically valid. In various embodiments, the exoskeleton system 100 can include sensors 513 embedded in the exoskeleton device 510 and/or pneumatic system 520 to provide a fuller picture of the system posture. In yet another embodiment, the exoskeleton system 100 can include logical constraints that are unique to the application in an effort to provide additional constraints on the operation of the pose estimation. This can be desirable, in some embodiments, in conditions where ground truth information is unavailable such as highly dynamic actions, where the exoskeleton system 100 is denied an external GPS signal, or the earth's magnetic field is distorted.

In some embodiments, changes in configuration of the exoskeleton system 100 based on location and/or location attributes can be performed automatically and/or with input from the user 101. For example, in some embodiments, the exoskeleton system 100 can provide one or more suggestions for a change in configuration based on location and/or location attributes and the user 101 can choose to accept such suggestions. In further embodiments, some or all configurations of the exoskeleton system 100 based on location and/or location attributes can occur automatically without user interaction.

Various embodiments can include the collection and storage of data from the exoskeleton system 100 throughout operation. In one embodiment, this can include the live streaming of the data collected on the exoskeleton device 510 to a cloud storage location via the communication unit(s) 514 through an available wireless communication protocol or storage of such data on the memory 512 of the exoskeleton device 510, which may then be uploaded to another location via the communication unit(s) 514. For example, when the exoskeleton system 100 obtains a network connection, recorded data can be uploaded to the cloud at a communication rate that is supported by the available data connection. Various embodiments can include variations of this, but the use of monitoring software to collect and store data about the exoskeleton system 100 locally and/or remotely for retrieval at a later time for an exoskeleton system 100 such as this can be included in various embodiments.

In some embodiments, once such data has been recorded, it can be desirable to use the data for a variety of different applications. One such application can be the use of the data to develop further oversight functions on the exoskeleton system 100 in an effort to identify device system issues that are of note. One embodiment can be the use of the data to identify a specific exoskeleton system 100 or leg actuator unit 110 among a plurality, whose performance has varied significantly over a variety of uses. Another use of the data can be to provide it back to the user 101 to gain a better understanding of how they ski. One embodiment of this can be providing the data back to the user 101 through a mobile application that can allow the user 101 to review their use on a mobile device. Yet another use of such device data can be to synchronize playback of data with an external data stream to provide additional context. One embodiment is a system that incorporates the GPS data from a companion smartphone with the data stored natively on the device. Another embodiment can include the time synchronization of recorded video with the data stored that was obtained from the device 100. Various embodiments can use these methods for immediate use of data by the user to evaluate their own performance, for later retrieval by the user to understand behavior from the past, for users to compare with other users in-person or through an online profile, by developers to further the development of the system, and the like.

Another aspect of a method of operating an exoskeleton system 100 can include monitoring software configured for identifying user-specific traits. For example, the exoskeleton system 100 can provide an awareness of how a specific skier 101 operates in the exoskeleton system 100 and over time can develop a profile of the user's specific traits in an effort to maximize device performance for that user. One embodiment can include the exoskeleton system 100 identifying a user-specific use type in an effort to identify the use style or skill level of the specific user. Through an evaluation of the user form and stability during various actions (e.g., via analysis of data obtained from the sensors 513 or the like), the exoskeleton device 510 in some examples can identify if the user is highly skilled, novice, or beginner. This understanding of skill level or style can allow the exoskeleton system 100 to better tailor control references to the specific user.

In further embodiments, the exoskeleton system 100 can also use individualized information about a given user to build a profile of the user's biomechanic response to the exoskeleton system 100. One embodiment can include the exoskeleton system 100 collecting data regarding the user to develop an estimate of the individual user's knee strain in an effort to assist the user with understanding the burden the user has placed on his legs 102 throughout use. This can allow the exoskeleton system 100 to alert a user if the user has reached a historically significant amount of knee strain to alert the user that he may want to stop to spare himself potential pain or discomfort.

Another embodiment of individualized biomechanic response can be the system collecting data regarding the user to develop an individualized system model for the specific user. In such an embodiment the individualized model can be developed through a system ID (identification) method that evaluates the system performance with an underlying system model and can identify the best model parameters to fit the specific user. The system ID in such an embodiment can operate to estimate segment lengths and masses (e.g., of legs 102 or portions of the legs 102) to better define a dynamic user model. In another embodiment, these individualized model parameters can be used to deliver user specific control responses as a function of the user's specific masses and segment lengths. In some examples of a dynamic model, this can help significantly with the device's ability to account for dynamic forces during highly challenging activities.

In various embodiments, the exoskeleton system 100 can provide for various types of user interaction. For example, such interaction can include input from the user 101 as needed into the exoskeleton system 100 and the exoskeleton system 100 providing feedback to the user 101 to indicate changes in operation of the exoskeleton system 100, status of the exoskeleton system 100, and the like. As discussed herein, user input and/or output to the user can be provided via one or more user interface 515 of the exoskeleton device 510 or can include various other interfaces or devices such as a smartphone user device. Such one or more user interfaces 515 or devices can be located in various suitable locations such as on a backpack 155 (see e.g., FIG. 1), the pneumatic system 520, leg actuation units 110, or the like.

The exoskeleton system 100 can be configured to obtain intent from the user 101. For example, this can be accomplished through a variety of input devices that are either integrated directly with the other components of the exoskeleton system 100 (e.g., one or more user interface 515), or external and operably connected with the exoskeleton system 100 (e.g., a smartphone, wearable device, remote server, or the like). In one embodiment, a user interface 515 can comprise a button that is integrated directly into one or both of the leg actuation units 110 of the exoskeleton system 100. This single button can allow the user 101 to indicate a variety of inputs. In another embodiment, a user interface 515 can be configured to be provided through a torso-mounted lapel input device that is integrated with the exoskeleton device 510 and/or pneumatic system 520 of the exoskeleton system 100. In one example, such a user interface 515 can comprise a button that has a dedicated enable and disable functionality; a selection indicator dedicated to the user's desired power level (e.g., an amount or range of force applied by the leg actuator units 110); and a selector switch that can be dedicated to the amount of predictive intent to integrate into the control of the exoskeleton system 100. Such an embodiment of a user interface 515 can use a series of functionally locked buttons to provide the user 101 with a set of understood indicators that may be required for normal operation in some examples. Yet another embodiment can include a mobile device that is connected to the exoskeleton system 100 via a Bluetooth connection or other suitable wired or wireless connection. Use of a mobile device or smartphone as a user interface 515 can allow the user a far greater amount of input to the device due to the flexibility of the input method. Various embodiments can use the options listed above or combinations and variants thereof, but are in no way limited to the explicitly stated combinations of input methods and items.

The one or more user interface 515 can provide information to the user 101 to allow the user to appropriately use and operate the exoskeleton system 100. Such feedback can be in a variety of visual, haptic and/or audio methods including, but not limited to, feedback mechanisms integrated directly on one or both of the actuation units 110; feedback through operation of the actuation units 110; feedback through external items not integrated with the exoskeleton system 100 (e.g., a mobile device); and the like. Some embodiments can include integration of feedback lights in the actuation units 110 of the exoskeleton system 100. In one such embodiment, five multi-color lights are integrated into the knee joint 125 or other suitable location such that the user 101 can see the lights. These lights can be used to provide feedback of system errors, device power, successful operation of the device, and the like. In another embodiment, the exoskeleton system 100 can provide controlled feedback to the user to indicate specific pieces of information. In such embodiments, the exoskeleton system 100 can pulse the joint torque on one or both of the leg actuation units 110 to the maximum allowed torque when the user changes the maximum allowable user-desired torque, which can provide a haptic indicator of the torque settings. Another embodiment can use an external device such as a mobile device where the exoskeleton system 100 can provide alert notifications for device information such as operational errors, setting status, power status, and the like. Types of feedback can include, but are not limited to, lights, sounds, vibrations, notifications, and operational forces integrated in a variety of locations that the user 101 may be expected to interact with including the actuation units 110, pneumatic system 520, backpack 155, mobile devices, or other suitable methods of interactions such as a web interface, SMS text or email.

The communication unit 514 can include hardware and/or software that allows the exoskeleton system 100 to communicate with other devices, including a user device, a classification server, other exoskeleton systems 100, or the like, directly or via a network. For example, the exoskeleton system 100 can be configured to connect with a user device, which can be used to control the exoskeleton system 100, receive performance data from the exoskeleton system 100, facilitate updates to the exoskeleton system, and the like. Such communication can be wired and/or wireless communication.

In some embodiments, the sensors 513 can include any suitable type of sensor, and the sensors 513 can be located at a central location or can be distributed about the exoskeleton system 100. For example, in some embodiments, the exoskeleton system 100 can comprise a plurality of accelerometers, force sensors, position sensors, and the like, at various suitable positions, including at the arms 115, 120, joint 125, actuators 130 or any other location. Accordingly, in some examples, sensor data can correspond to a physical state of one or more actuators 130, a physical state of a portion of the exoskeleton system 100, a physical state of the exoskeleton system 100 generally, and the like. In some embodiments, the exoskeleton system 100 can include a global positioning system (GPS), camera, range sensing system, environmental sensors, elevation sensor, microphone, thermometer, or the like. In some embodiments, the exoskeleton system 100 can obtain sensor data from a user device such as a smartphone, or the like.

In some cases, it can be beneficial for the exoskeleton system 100 to generate or augment an understanding of a user 101 wearing the exoskeleton system 100 of the environment and/or operation of the exoskeleton system 100 through integrating various suitable sensors 515 into the exoskeleton system 100. One embodiment can include sensors 515 to measure and track indicators to observe various suitable aspects of user 101. These indicators can include biological indicators such as body temperature, heart rate, respiratory rate, blood pressure, blood oxygenation saturation, expired CO₂, blood glucose level, sweat rate, muscle activation, EMG, EKG, muscle fatigue, joint rotational speeds and accelerations, and the like and performance indicators such as balance, agility, gait speed, time to complete a physical task, time to complete a cognitive task and the like.

In some embodiments, the exoskeleton system 100 can take advantage of the relatively close and reliable connectivity of such sensors 515 to the body of the user 101 to record system vitals and store them in an accessible format (e.g., at the exoskeleton device, a remote device, a remote server, or the like). Another embodiment can include environmental sensors 515 that can continuously or periodically measure the environment around the exoskeleton system 100 for various environmental conditions such as temperature, humidity, light level, barometric pressure, radioactivity, sound level, toxins, contaminants, or the like. In some examples, various sensors 515 may not be required for operation of the exoskeleton system 100 or directly used by operational control software, but can be stored for reporting to the user 101 (e.g., via an interface 515) or sending to a remote device, a remote server, or the like.

The pneumatic system 520 can comprise any suitable device or system that is operable to inflate and/or deflate the actuators 130 individually or as a group. For example, in one embodiment, the pneumatic system can comprise a diaphragm compressor as disclosed in related patent application Ser. No. 14/577,817 filed Dec. 19, 2014, or a pneumatic power transmission as discussed herein.

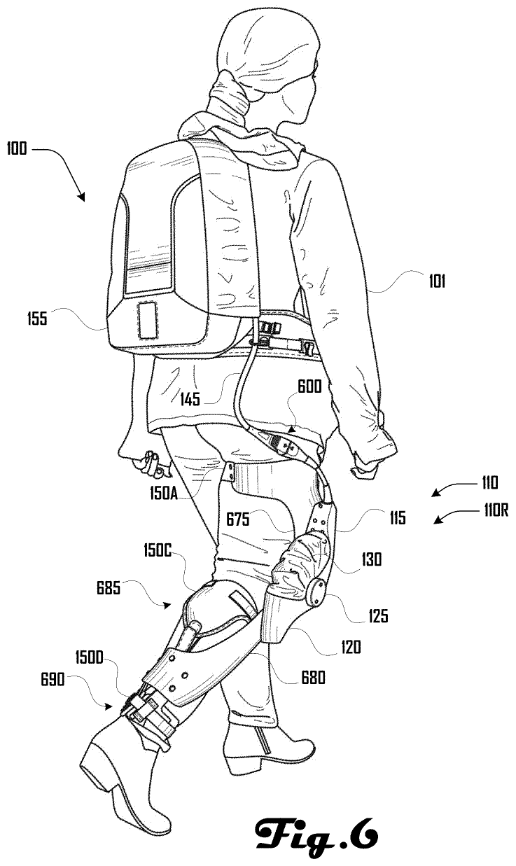

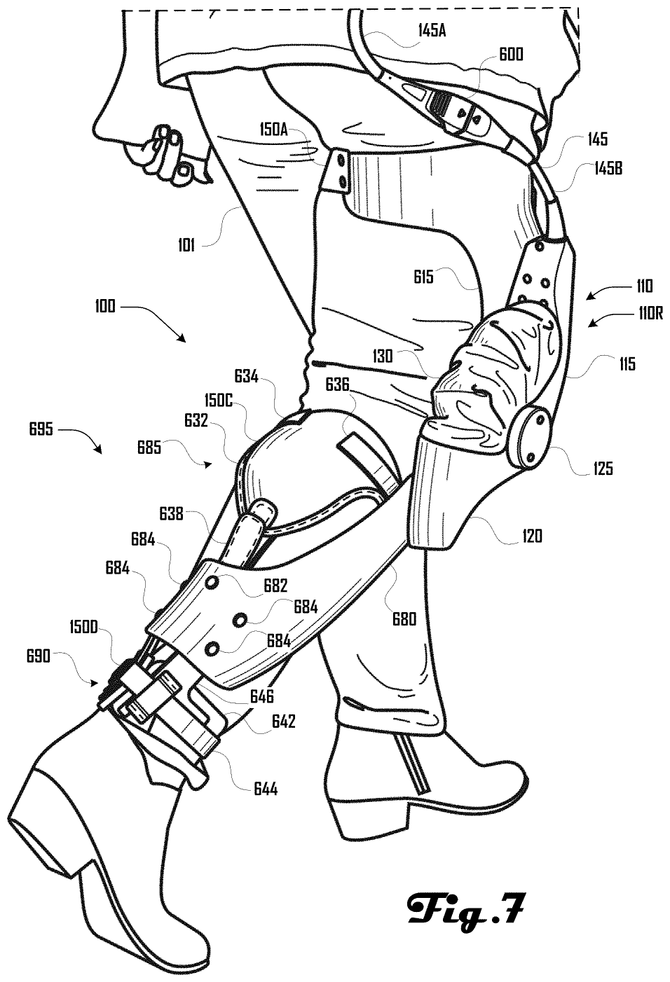

Turning to FIGS. 6 and 7, another embodiment of an exoskeleton system 100 is illustrated. In this example embodiment, the exoskeleton system 100 includes a single right leg actuator unit 110; however, it should be clear that this example embodiment can be extended to an exoskeleton system 100 having both a left and right actuator unit 110L, 110R or only a left actuator unit 110L. Accordingly the example of FIGS. 6 and 7 should not be construed as limiting, and in further embodiments, any suitable elements can be present in a suitable plurality, absent, or interchanged with elements of other embodiments (e.g., FIGS. 1-4), or the like.

As shown in FIGS. 6 and 7, the leg actuator unit 110 can include an upper arm 115 and a lower arm 120 that are rotatably coupled via a joint 125. A bellows actuator 130 extends between the upper arm 115 and lower arm 120. A cable 145 can be coupled to the bellows actuator 130 to provide power, communication and/or introduce and/or remove fluid from the bellows actuator 130 to cause the bellows actuator 130 to expand and contract and to stiffen and soften, as discussed herein. As shown in the example of FIGS. 6 and 7, the cable 145 can comprise a cable connector 600 that can define a releasable coupling along a portion of the cable 145 with first and second cable portions 145A, 145B on opposing sides of the cable connector 600. As discussed in more detail herein, in various embodiments, the cable connector 600 can provide for a releasable coupling of a unified unitary cable 145 that comprises elements for fluid transfer, electrical power transfer and/or communications to, from or between the exoskeleton device 510 (e.g., disposed in the backpack 155) and an exoskeleton unit 110 and/or actuator 130 as discussed herein. In some embodiments, the cable connector 600 can couple directly with the exoskeleton device 510 (e.g., disposed in the backpack 155) or exoskeleton unit 110 and/or actuator 130 such that only a single cable portion 145A or 145B extends from the cable connector 600.

A backpack 155 can be worn by the user 101 (see FIG. 6) and can hold various components of the exoskeleton system 100 such as a fluid source, control system, a power source, exoskeleton device, pneumatic system, and the like as discussed herein. For example, in some embodiments, the backpack 155 can comprise or store one or more of the components of an exoskeleton device 510 (see e.g., FIG. 5).

As shown in FIGS. 6 and 7, the leg actuator unit 110 can be coupled about the right leg of the user 101 with the joint 125 positioned at the right knee 103R of the user 101 (see FIGS. 1-3 for labeling of body parts of the user 101), with the upper arm 115 of the leg actuator unit 110R being coupled about the right upper-leg portion 104R of the user 101 via one or more couplers 150 (e.g., straps that surround the legs 102). The lower arm 120 of the leg actuator unit 110 can be coupled about the right lower-leg portion 105R of the user 101 via one or more couplers 150.

The upper and lower arms 115, 120 of a leg actuator unit 110 can be coupled about the leg 102 of a user 101 in various suitable ways. For example, FIGS. 6 and 7 illustrate an example where the upper and lower arms 115, 120 and joint 125 of the leg actuator unit 110 are coupled along lateral faces (sides) of the top and bottom portions 104, 105 of the leg 102. As shown in the example of FIGS. 6-9, the upper arm 115 can be coupled to the upper-leg portion 104 of a leg 102 above the knee 103 via one coupler 150 and the lower arm 120 can be coupled to the lower-leg portion 105 of a leg 102 below the knee 103 via two couplers 150.

Specifically, the upper arm 115 can be coupled to the upper-leg portion 104 of the leg 102 above the knee 103 via a first upper-leg coupler 150A. The first upper-leg coupler 150A can be associated with a rigid upper-leg brace 675 disposed on and engaging a lateral side of the upper-leg portion 104 of the leg 102, with a strap of the first upper-leg coupler 150A extending around the upper-leg portion 104 of the leg 102. The upper arm 115 can be coupled to the rigid upper-leg brace 675 on a lateral side of the upper-leg portion 104 of the leg 102, which can transfer force generated by the actuator 130 through the upper arm 115 to the upper-leg portion 104 of the leg 102.

The lower arm 120 can be coupled to the lower-leg portion 105 of a leg 102 below the knee 103 via a second set of couplers 695 that includes first and second lower-leg couplers 150C, 150D. The first and second lower-leg couplers 150C, 150D can be associated with a rigid lower-leg brace 680 disposed on and engaging a lateral side of the lower-leg portion 105 of the leg 102. The lower arm 120 can be coupled to the rigid lower-leg brace 680 on a lateral side of the lower-leg portion 105 of the leg 102, which can transfer force generated by the actuator 130 through the lower arm 120 to the lower-leg portion 105 of the leg 102. The rigid lower-leg brace 680 can extend downward from a coupling with the lower arm 120 at a lateral position on the lower-leg portion 105 of the leg 102, with a portion of the rigid lower-leg brace 680 curving toward the posterior (back) of the lower-leg portion 105 to attachments 682, 684 that couple one or more portions of the first and second lower-leg couplers 150C, 150D to the rigid lower-leg brace 680.

The first lower-leg coupler 150C can include a calf-coupling assembly 685 that includes a calf brace 632 that is coupled to the rigid lower-leg brace 680 via a first, second and third calf strap 634, 636, 638. For example, as shown in the example of FIGS. 6 and 7, the first and second calf straps 634, 636 can extend horizontally from opposing lateral sides of an upper portion of the rigid lower-leg brace 680 from an internal face of the rigid lower-leg brace 680. The third calf strap 638 can extend vertically from a lower posterior portion of the rigid lower-leg brace 680 from an internal face of the rigid lower-leg brace 680 where the third calf strap 638 is coupled to the rigid lower-leg brace 680 via a first set of one or more attachments 682. In various embodiments, the calf brace 632 can be a rigid or flexible element and can comprise materials such as a fabric, plastic, carbon-fiber, or the like. These examples in no way limit the possible configurations of the calf strap, including the number of straps, which may vary from 1, 2, 3, 5, 6, 10 and the like; their extension direction from the upper portion and/or lower portion of the rigid lower-leg brace 680; and whether they extend from an internal or external face or edge of the rigid lower-leg brace 680.

The calf straps 634, 636, 638 can be configured in various suitable ways and can include various suitable mechanisms that allow the calf straps 634, 636, 638 to be tightened, loosened, extended, shortened, removed, or the like. For example, in some embodiments, the first and second calf straps 634, 636 comprise hook and loop tape (e.g., Velcro) that allows the second calf straps 634, 636 to be tightened, loosened, extended, shortened, or the like. In some embodiments, the third calf strap 638 can comprise a strap cinch, or the like, that allows the third calf strap 638 to be tightened, loosened, extended, shortened, or the like.

The second lower-leg coupler 150D can comprise an ankle-coupling assembly 690 that includes a cuff 642 that extends around and surrounds the lower-leg portion 105 in proximity to the ankle of the user 101, including on, above or below the ankle within 0 mm, 6 mm, 1 cm, 5 cm, 10 cm, and held via an ankle strap 644. The cuff 642 can be coupled to the rigid lower-leg brace 680 via one or more coupling tabs 646 that extend vertically from the cuff 642, with the one or more coupling tabs 646 coupled to the rigid lower-leg brace 680 via a second set of one or more attachments 64 on an internal face of the rigid lower-leg brace 680. In some embodiments, the coupling tab 646 is fixed relative to the rigid lower-leg brace 680, which in turn fixes the position of the ankle-coupling assembly 690 relative to the rigid lower-leg brace 680. In other embodiments, the coupling tab is semi-rigidly fixed to the rigid lower-leg brace 680, allowing for adjustment of the ankle-coupling assembly 690 position relative to the rigid lower-leg brace 680. In some embodiments of this, the adjustment is done manually, such as by loosening and tightening an adjustment screw, by the user, someone trained in the fitting of the device to the user, or another person and the like, or the adjustment is controlled by the exoskeleton system through such means as a rack and pinion gear driven by a motor and the like. In other embodiments, the coupling tab remains free to move relative to the rigid lower-leg brace 680, allowing for dynamic adjustment of the ankle-coupling assembly 690 position relative to the rigid lower-leg brace 680 which can accommodate the movements of the user. The ankle strap 644 can include various suitable elements that allow the ankle strap to be tightened, loosened, extended, shortened, removed or the like (e.g., hook and loop tape, strap cinch, or the like).

In various embodiments, the rigid upper-leg and lower-leg braces 675, 680 can be made of various suitable materials such as a plastic, carbon-fiber, metal, wood, or the like. As discussed herein, in some embodiments the upper-leg and/or lower-leg braces 675, 680 can be formed to match the contours of the legs 102 of the user 101, which can be desirable for increasing comfort for the user 101 maximizing surface area of the upper-leg and/or lower-leg braces 675, 680 engaging the legs 102 of the user 101, and the like. In some examples, the upper-leg and/or lower-leg braces 675, 680 can be formed specifically for a given user 101, which can include molding to user body parts, scanning the user's body and generating upper-leg and/or lower-leg braces 675, 680 from such scan data, and the like. In some examples, the upper-leg and/or lower-leg braces 675, 680 can be formed specifically for a given set of users 101, such as those with similar body morphologies such that they can be used to fit segments of the user population.

In some embodiments, alignment and suspension of one or more actuation units 110 on the leg 102 (or other body parts) of a user 101 can be achieved in some examples via a strap connected at the lower-leg 105 just above the ankle of the user 101. For example, such a strap can be firmly placed in a supra-malleolar location that is located above the malleolus (protruding bones at the ankle) and below the bulk of the calf muscle. Such a strap can be connected in a firm connection such that it lies in a narrowing diameter portion of the user's leg 102. For example, coupler 150D of FIGS. 1-4, 6 and 7 and/or ankle coupling assembly 690 of FIGS. 6 and 7 can be configured in such a way. Such a connection method can be beneficial in some examples by having no portion of the actuator unit 110 (or at least no substantive portion used for coupling) extending below the ankle of the user 101 to interface with the user's foot, user's footwear, the ground, or area below the malleolus. In some examples, where the user's footwear extends to a supra-malleolar location, it can be advantageous to interact with the footwear, the advantages including but not limited to improving comfort, reducing irritation, increasing friction and suspension of the actuation unit 110, reducing the accuracy needed in the location of the ankle coupling assembly 690 on the lower-leg 105, and the like.

While various embodiments discussed and illustrated herein can relate to exoskeleton systems 100 configured for users 101 having all conventional body parts, further embodiments can include exoskeleton systems configured to be worn by users 101 that are amputees or persons who otherwise do not have all conventional body parts (e.g., a person who is missing one or more toe, foot, lower leg, leg, knee joint, finger, hand, distal portion of an arm, elbow joint, arm, or the like).

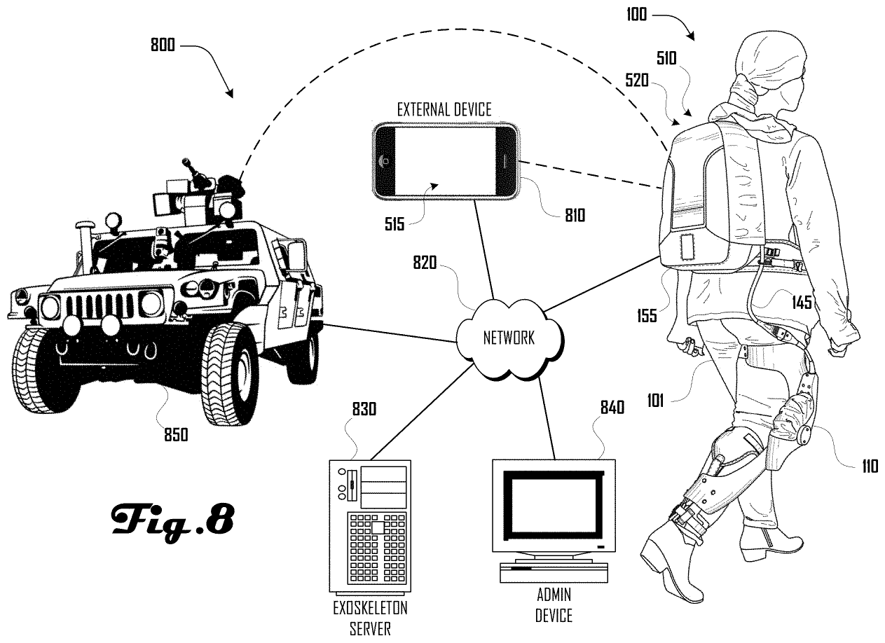

Turning to FIG. 8, an embodiment of an exoskeleton network 800 is illustrated that includes an exoskeleton system 100 that is operably coupled to an external device 810 and a vehicle 850 via a direct connection and/or via a network 820. The exoskeleton system 100 can also be operably coupled to an exoskeleton server 830 and an admin device 840 as illustrated in the example of FIG. 8. For example, in some embodiments, some or all of the exoskeleton device 510 and/or pneumatic system 520 (see also FIG. 5) can be disposed within a backpack 155 configured to be worn by the user 101, and the exoskeleton device can be operably connected to an external device 810, vehicle 850 and/or network 820 via a communication unit 514 of the exoskeleton device 510 (see FIG. 5). Such one or more connections can be wireless and/or wired connections of various suitable types, such as Bluetooth, RFID, Wi-Fi, a cellular connection, a radio connection, a microwave connection, a satellite connection, or the like.

In some embodiments, the exoskeleton system 100 can be operably connected to the network (and the server 830, admin device 840 and/or vehicle 850) via the external device 810. For example, the exoskeleton device 510 may not have a direct operable connection to the network 820 and, instead, can have a direct connection to the external device 810 and the external device 810 has an operable connection to the network 820, which allows the exoskeleton system 100 to communicate with the network (and the server 830, admin device 840 and/or vehicle 850) via the external device 810.

The network 820 can comprise any suitable wired and/or wireless network such as the Internet, a satellite network, a cellular network, a military network, a microwave network, a Wi-Fi network, a Large Area network (LAN), a Wide Area Network (WAN), or the like. Additionally, the example of FIG. 8 should not be construed as being limiting and any of the illustrated elements can be specifically absent or present in any suitable plurality in further embodiments. For example, in some embodiments, a plurality of exoskeleton systems 100 can be connected to the network 820, which can allow for communication between or among the plurality of exoskeleton systems 100.

The external device 810 in the example of FIG. 8 is show as comprising a smartphone, but various other suitable external devices can be used in further embodiments, including a tablet computer, a headset device, a smartwatch, an embedded system, or the like. In various examples, the external device 810 can present a user interface 515 that allows input and/or feedback as discussed herein. However, it should be noted that the presence of a user interface 515 of an external device 810 does not mean that one or more additional user interfaces 515 is not present on or in the exoskeleton network 800 or exoskeleton system 100. For example, as discussed herein, one or more user interfaces 515 can be located in various suitable locations such as on, in or about a backpack 155, at one or more leg actuation units 110, at a pneumatic line 145, or the like. Additionally, while the example of a vehicle 850 is shown in FIG. 8, in further embodiments the exoskeleton system 100 can be operably connected to various vehicles or systems such as a ship, boat, submarine, truck, car, oil rig, dock, building, airplane, jet, or the like.

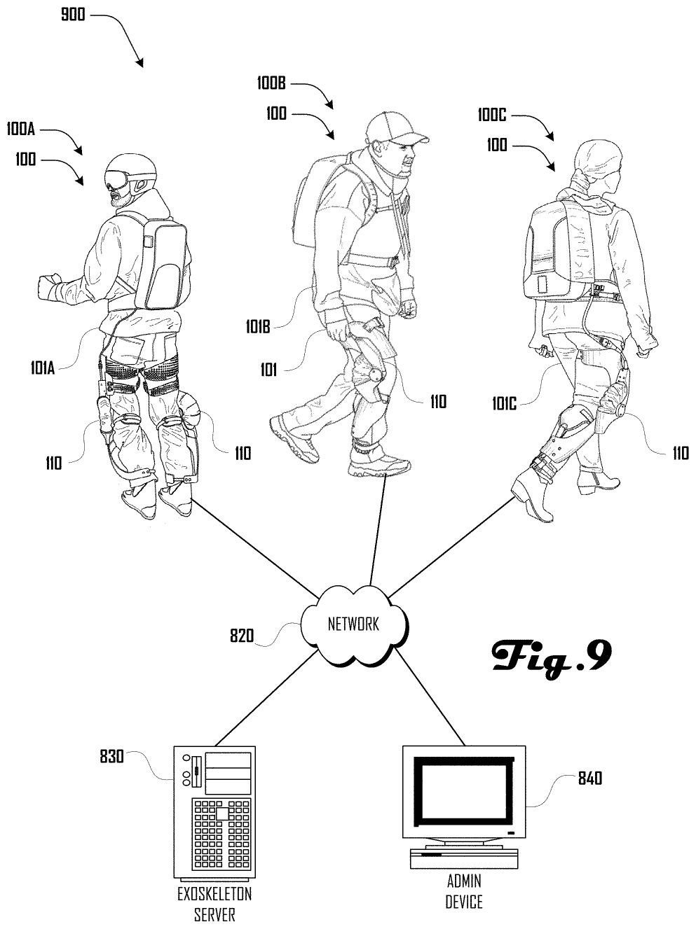

Turning to FIG. 9, another embodiment of an exoskeleton network 900 is illustrated which comprises a plurality of exoskeleton systems 100 being worn by a plurality of respective users 101, with the plurality of exoskeletons being operably connected to an exoskeleton server 630 and an admin device 640 via a network 620. Specifically, a first, second and third exoskeleton system 100A, 100B, 100C are being respectively worn by a first, second and third user 101A, 101B, 101C. While the example of FIG. 9 illustrates three exoskeleton systems 100 and three users 101, further embodiments can include any suitable plurality of exoskeleton systems 100 and users 101, including 2, 3, 4, 5, 6, 7, 8, 9, 10, 20, 30, 50, 100, 150, 250, 500, 1000, 10000, and the like.

In some cases, a plurality of users 101 wearing exoskeleton systems 100 can be present together in a group such as a team of soldiers, law enforcement, first responders, recreational users, workers or the like. For example, in one embodiment, a group of skiers wearing skiing exoskeleton systems 100 can be together on a slope or ski area. In some cases, one or more of a plurality of exoskeleton systems 100 can be remote from each other. For example, a plurality of users 101 can operate an exoskeleton system 100 at their homes, at work, on vacation, or the like, and may be located in disparate physical locations such as in different countries, states, counties, cities, areas, or the like.

One aspect of software being executed by one or more exoskeleton systems 100 (e.g., by an exoskeleton device 510) can include the collection of data from the one or more exoskeleton systems 100 during operation. For example, one embodiment of an exoskeleton device 510 can store data (e.g., in memory 512) including data obtained from one or more sensors 513 of the exoskeleton system 100; references generated by the exoskeleton device 510 for configuring one or more actuation units 110 (e.g., torque, pressure, or angle references); determinations of intended or current maneuvers (e.g., sitting, standing, walking, running, crouching, jumping, and the like); determination of a state transition (e.g., going from sitting to standing, going from standing to sitting, going from walking to running, going from standing to jumping, and the like); determination of environmental conditions (e.g., ground slope, terrain type, temperature, air pressure, and the like); determination of location data (e.g., GPS coordinates, elevation, location of other devices or exoskeleton system 100, and the like); electric power status (e.g., charge state of one or more batteries, removal or attachment of one or more batteries, power consumption, and the like); fluid status (e.g., amount of fluid present in a fluid source, amount of fluid introduced to or released from one or more leg actuation units 110, and the like); commands to any component of the exoskeleton system 100 including to the leg actuation units 110 such as opening or closing a fluidic valve or to the pneumatic system 520 such as turning on or off a pneumatic compressor; a determined physical configuration of the user 101 and/or exoskeleton system 100 (e.g., a model of a determined configuration of the user 101 based on sensor data; a model of the configuration of one or more actuation units 110 based on sensor data, and the like); system error data; input and/or feedback associated with one or more user interfaces 515, and the like.

In various embodiments, such data can be associated with location and/or time data, which can be desirable for context of when and where such data was obtained, determined, or the like. For example, a plurality of data units of a set of exoskeleton data can be respectively associated with different time and/or location data corresponding to a location of the respective exoskeleton system when a given data unit was generated or obtained and/or a time when the data unit was generated or obtained.

In various embodiments, such data can be associated with a user ID and/or exoskeleton ID. For example, a plurality of data units of a set of exoskeleton data can be associated with a different user ID and/or exoskeleton ID corresponding to a user 101 or exoskeleton system 100 associated with a respective data unit. Such one or more ID can be desirable so that data from different users 101 and/or exoskeleton systems 100 can be differentiated, which can allow for customized configuration input, data visualization, or the like, for a given user 101 and/or exoskeleton system 100.

In some examples, a given user 101 can use a specific exoskeleton system 100 and data generated during use of the specific exoskeleton system 100 by the given user 101 can be associated with the given user 101 and the specific exoskeleton system 100. This association between the data, a given user 101 and/or the specific exoskeleton system 100 can be accomplished in a number of ways, including but not limited to the user connecting their user ID to the specific exoskeleton system 100 prior to, during or after use by logging into the specific exoskeleton system 100, the user ID and exoskeleton ID being recorded together through digital means, as in a video recording, spreadsheet or logfile for example, or manual means, as on a sheet of paper, and the like. Such a configuration can be desirable so that data from specific users 101 and exoskeleton systems 100 can be differentiated, such as when a given user 101 operates a plurality of different exoskeleton systems 100 or when a plurality of users 101 use a given exoskeleton system 100. Such differentiation can allow characteristics of a given user 101 to be tracked regardless of the exoskeleton system 100 that the user 101 is operating. Similarly, such differentiation can allow characteristics of an exoskeleton system 100 to be tracked regardless of the users 101 that are using the exoskeleton system 100.

In various embodiments, a user ID may be associated with the biometric, biomechanical, genetic, and/or other identifying data related to a specific user 101. This data can include, but is not limited to, the user's height, weight, gender, ethnicity, age, body fat percentage, eye color, hair color, torso length, thigh length, shank length, distance of ankle joint from the ground, distance of ankle joint to other joints, range of motion at the knee and other joints, fingerprint scan, retinal scan, weight lifting ability (e.g., maximum bench press, squat), dynamic mobility (e.g., 100 m sprint), endurance (e.g., time to complete 5 k run), ability to complete a puzzle within a given time period or other cognitive task, other physical or cognitive performance metrics, lung capacity, body temperature, and the like.

In some embodiments, the user identifying data associated with a specific user 101 is used to select an initial configuration of the exoskeleton system 100 for the user 101 before the user's first time ever using the exoskeleton system 100. This initial configuration may include a physical configuration, such as limiting a range of motion at the knee joint of a left leg actuator unit 110L based on the user's range of motion at the left knee joint. This physical configuration may be manually set by the user 101 or other person, or it may be automatically set by the exoskeleton system 100. The initial configuration may include an initial software configuration selected based on the user identifying data, with preset references, thresholds, control algorithms, and the like.

Also, in some embodiments, the user identifying data associated with a plurality of users 101 can be correlated with data collected from exoskeleton systems 100 used by users 101. These correlations can be used in a variety of ways, including, but not limited to, informing an initial configuration of the exoskeleton system 100 for a user 101 based on the user's identifying data (e.g., the user's height and weight correlated to a length of left leg actuator unit 110L), changing the configuration of the exoskeleton system 100 for a user 101 based on a correlation between exoskeleton system 100 performance and the user's identifying data (e.g., a rehabilitation configuration for a user 101 with poor range of motion, a dynamic assistance configuration for a user with a fast 100 m time, higher scaled assistance levels for a heavier user), and the like.

Also, in some embodiments, such data can also be associated with or comprise a confidence level. For example, where user intent to transition from a sitting position to a standing position is identified and stored as data, such data can be associated with a confidence level at which such user intent was identified (e.g., 10%, 50%, 80%, or the like).

Also, some embodiments can include an identification of whether a determination made by the exoskeleton system was correct or incorrect, which can also include a confidence level. For example, where user intent to transition from a sitting position to a standing position is identified as discussed in the example above, a determination can be made whether such an intent recognition was correct; in other words, whether the user was actually transitioning from a sitting position to a standing position or whether such a determination was an incorrect false-positive.

Various embodiments can include the streaming of such data collected on the exoskeleton device 510 to an external and/or remote device such as an external device 610, exoskeleton server 630, admin device 640, one or more other exoskeleton systems 100, or the like (see FIGS. 6 and 7). For example, exoskeleton data can be communicated to a secure cloud storage location (e.g., exoskeleton server 630) through an acceptable wireless communication protocol (e.g., via the network 620). In some embodiments, this data communication can occur simultaneously or after a given delay after its collection such as 1 microsecond, 1 millisecond, 0.01 seconds, 1 second, 1 minute, 1 hour, and the like. In some embodiments, an exoskeleton system 100 may operate in a connection-limited environment and can store data onboard the exoskeleton device 510 (e.g., in memory 512) and then communicate such data when desirable or when a connection becomes available.

For example, an exoskeleton system 100 can operate in a location where a connection to the network 620 is unavailable, and when the exoskeleton system 100 reaches a location where a connection is available, the exoskeleton system 100 can use a high-bandwidth secured connection protocol to upload large amounts of data to a secure cloud location (e.g., exoskeleton server 630) via the network 620 at a communication rate that is supported by the available data connection, or upload such data to a local storage device (e.g., external device 610), which can then be uploaded to the a remote server such as an exoskeleton server 630.

In various embodiments, the exoskeleton system 100 can choose when to upload data to an external or remote device; however, in some embodiments, one or more of such devices can request or cause such data to be uploaded. For example, an admin device 640 can request or cause data stored on an exoskeleton device 510 to be uploaded to an exoskeleton server 630 or can turn on or turn off real-time data reporting by the exoskeleton device 510.

Once exoskeleton data has been stored either locally on the exoskeleton device 510 or on an external device 610, exoskeleton server 630, or the like, such exoskeleton data can be used for a variety of different applications. One such application is the use of the exoskeleton data to develop oversight functions for the exoskeleton system 100 or the user 101 of the exoskeleton system 100 in an effort to identify system issues that are of note. One embodiment is the use of the data to identify one or more exoskeleton systems 100 whose performance has varied significantly over a variety of uses, which can be used to identify a need for maintenance, generate a software or firmware update, or the like.

Data from sensors and other relevant data can be used by the exoskeleton system 100 in various examples (or other device such as an external device 810, exoskeleton server 830, admin device 840 and the like) to make inferences about the physical and physiological states of the user 101 with such data being obtained from sensors of the exoskeleton system 100, an external device 810, exoskeleton server 830, admin device 840 and the like. Such inferences may be made about a past state of the user 101, the immediate state of the user 101, the future state of the user 101, the cause of a past or immediate state of the user 101, the likelihood of a future state of the user 101, and the like. In one embodiment, data from sensors of the exoskeleton system 100 (e.g., sensors 513 of the exoskeleton device 510 or sensors disposed on the actuator unit(s) 110 or the like) can be used to detect when a user has fallen. In one example embodiment, acceleration of the torso and legs of the user 101 towards the ground could indicate the user 101 has experienced a fall event. In other embodiments, a method to infer a fall may include, but is not limited to, using one or more accelerometers to measure impact with the ground, using one or more IMUs (e.g., accelerometer, gyroscope, magnetometer), barometers, and encoders to estimate the body pose of user in a prone, supine, similar lying position, or the combination of previously mentioned methods, and the like. In another embodiment, the exoskeleton system 100 may record sensor data leading up to and including previous fall events, such as the direction or magnitude of impact with the ground, time spent walking or moving, types of activities the user engaged in, time spent on the ground, location, altitude, weather, and the like, and use that data from previous events to infer the probability of a future fall event given the current sensor data. In another embodiment, it can be inferred from immediate sensor data that a user 101 is in a state of physical fatigue, such as from a diminished knee angle velocity, altered gait, or reduced muscle activity recorded by EMG, which could then also provide inference of the increased probability of a future fall event.

In one embodiment, measuring an acceleration whose magnitude is greater than gravitational acceleration (approximately 9.8 m/s\hat{ }2 and hereby referred to as a g-force), 2 g-forces, 8 g-forces, 20 g-forces, and the like, of the user 101 or any part of the user 101 can provide the basis for one or more inferences in various embodiments, including but not limited to: the user 101 has been exposed to an explosion or blast, the existence of an explosion or blast, has been struck by another human or object, has experienced a collision with an external object, such as but not limited to a vehicle, building, other user, or the ground, is currently experiencing impacts, is in a flight phase due to an impact or exposure to an explosion, is in a vehicle, that the vehicle is experiencing impacts from another vehicle, a building, a human, an object, terrain, or encountered an explosion, and the like. In one example embodiment, such (high) accelerations can be used to infer that the environment surrounding the user is dangerous, such as a minefield or a terrain exposed to ballistic or projectile fire, or that there are dangerous elements, such as enemy combatants, nearby, allowing for the alerting of the user, surrounding users, and/or a user at a distance, to the situation.

For example, a method of data inference of an exoskeleton system 100 can comprise obtaining sensor data indicating an acceleration of the user 101 and/or exoskeleton system 100 greater than 1 g-force (e.g., from sensor(s) 513 of an exoskeleton device 510, sensor(s) associated with one or more actuator units 110, or the like); determining based at least in part on this sensor data one or more condition or state of the user 101, condition or state of the exoskeleton system 100 and/or condition or state of a location or environment where the user 101 and/or exoskeleton system 100 is present; and determining and implementing a response based on the determined one or more conditions. For example, such a response can include changing a configuration of the exoskeleton system 100, actuating the exoskeleton system 100, generating an alert by the exoskeleton system 100 (e.g., audio, visual, haptic), sending an alert to another devices (e.g., an external device 810, exoskeleton server 830, admin device 840), or the like.

In another embodiment, acceleration magnitudes less than one g-force in a direction perpendicular to the ground as measured by an accelerometer on the user 101 or any part of the user 101 can be used to infer that the user is in free-fall. Such one or more sensors can be sensor(s) 513 of an exoskeleton device 510, sensors associated with one or more actuator units 110, or the like. In response, for example, the exoskeleton system 100 can change the assistance level (torque) of one or more of actuator units 110 in anticipation of the landing from such a free-fall state.

For example, a method of operating an exoskeleton system 100 can include determining by the exoskeleton system 100 that the exoskeleton system 100 is in free fall or in a jump and determining to assist the user with a landing associated with the free fall or jump. For example, determining to assist the user 101 with the user action can be based at least in part on sensor data from the exoskeleton system 100, intent recognition based on the sensor data from the exoskeleton system 100 and indication by the user for a specific action or intent to perform an action (e.g., provided by the external device 810, user interface 515, button, or the like). In various embodiments, such a determination can be based at least in part on obtaining sensor data indicating acceleration magnitudes less than one g-force in a direction perpendicular to the ground, which can be associated with one or more joints of user (e.g., one or more actuator units 110) or associated with the body of a user generally. The exoskeleton system 100 can then actuate the actuator unit(s) 110 to assist the user with landing the free fall or jump.

In one embodiment, a user's exposure to weather conditions immediate to the user 101 and the exoskeleton system 100 such as cold, hot, dry, wet, and the like, can be tracked by sensors on the exoskeleton system 100, sensors on a group of exoskeleton system 100, provided to the system by a weather system or other external weather data source, and risk to the user 101 can be inferred. Some examples of sensors to measure a user's exposure to weather conditions can include, but are not limited to, one or more temperature sensors, anemometers, barometers, humidity sensors, water contact sensors, heart rate sensors, sweat sensors, accelerometers, and the like. The risk to the user may be an evaluation of how the weather may impact the user, such as, but not limited to, diminishing the user's health, restricting the user's ability to move, restricting the ability of other users or vehicles in reaching the user, the range of distance the user is capable of traveling, the danger of lightning striking the user, and the like.

Some example embodiments can include a method of operating an exoskeleton system 100 based at least in part on weather data, which can comprise obtaining weather data (e.g., from sensors of the exoskeleton system 100, from a location of the exoskeleton system 100 such as a vehicle 850, from an external device 810, exoskeleton server 830, admin device 840, or the like). In some embodiments, the method can include obtaining location data (e.g., GPS data); determining a location of the exoskeleton system 100 based at least in part on the location data; and obtaining the weather data based corresponding to the determined location of the exoskeleton system 100. Such a method can further include determining whether weather conditions are and/or are anticipated to be undesirable above or below a given threshold (e.g., temperature too high, temperature too low, lightening danger too high, rain, snow, or the like). If it is determined that the weather conditions are and/or are anticipated to be undesirable above or below the given threshold, a response determination can be made and implemented (e.g., operating the exoskeleton system 100 in a different operating parameters or in a different configuration based on such weather conditions, shutting down the exoskeleton system 100, providing an alert to the user 101 regarding the conditions, and the like).

In another embodiment, a user's exposure to water can be measured using sensors of the exoskeleton system 100 such as one or more accelerometers, gyroscopes, magnetometers, water contact sensors, humidity sensors, temperature sensors, skin conductance, sweat sensors, and the like. In some embodiments, the exoskeleton system 100 may have water sensors to detect the amount of water around the exoskeleton system 100 such as around the actuation unit(s) 110 and/or actuator(s) 130. As mentioned above, some or all of the exoskeleton system 100 may be waterproof or water resistant in some examples so it can operate in pools of water for short or long periods of time. Since it can be more difficult to move in pools of water or other liquid, in some embodiments the exoskeleton system 100 can be configured to increase the output strength of the actuator(s) 130 when a determination is made that a user wearing the exoskeleton system 100 is in a pool of water. In some embodiments, the exoskeleton system 100 may also use a pressure sensor, which may detect the pressure caused by the water and determine the amount of water and depth, to adjust and determine operation of the exoskeleton system 100 based at least in part on the determined presence of water and/or of the depth of the exoskeleton system within the water.

A method of determining actuation of exoskeleton system 100 based on water and/or depth data can include obtaining sensor data regarding the presence of water around the exoskeleton system 100 and/or or data indicating a depth of water that the exoskeleton system 100 is operating in. Such data can comprise data obtained from sensors associated with one or more actuation units 110, an exoskeleton device 510, or another suitable device with such sensors including a suitable water presence sensor, a suitable water depth sensor, or the like. In some embodiments, the exoskeleton system 100 can operate in a first configuration when it is determined that the exoskeleton system 100 is not fully or partially submerged in a body of water (e.g., that the one or more actuation units 110 are fully or partially submerged in water) and can be configured to operate in a second configuration different from the first configuration when it is determined that the exoskeleton system 100 is not fully or partially submerged in a body of water. For example, operating an actuation unit 110 in a body of water can result in greater resistance to movement and it can be desirable to increase power output by an actuator 130 to compensate for the increased resistance to provide for the same or substantially similar support and assistance while in water. Accordingly, in various embodiments, the first operating configuration for operation outside of water can provide for less power output by one or more actuator units 110 of an exoskeleton system 100, with the second operating configuration for operation within a body of water can provide for greater power output by the one or more actuator units 110 compared to the first operating configuration. In various examples, the exoskeleton system 100 can switch between the first and second operating configuration based on determinations of whether the exoskeleton system 100 is in a body of water or not in a body of water, which can be made at regular intervals or continuously.

In some embodiments, the parameters of the second operating configuration can be further based at least in part on a determined depth within the body of water. For example, resistance to movement of the actuation unit(s) 100 can increase as with depth within the body of water. Additionally, elements of the exoskeleton system 100 such as gas lines, actuators 130, operating gas, and the like, may be impacted by higher pressure at greater depths. For example, gas lines and the actuators 130 may be compressed at high depths and require additional pressure to achieve the same actuation force via the actuation unit(s) 110. Accordingly, a method can include obtaining water depth data, determining a suitable operating configuration based on the depth data, and configuring the exoskeleton system 100 accordingly based on the determined suitable operating configuration corresponding to the determined depth.

The exoskeleton system 100 in various embodiments may infer from sensor data that the user 101 may, is or has fallen from a vehicle into water, that the vehicle is submerged in water, and the like. For example, a method of operating an exoskeleton system 100 to prevent a user 101 from or assist a user falling from a vehicle can include obtaining data from the exoskeleton system 100 and/or another device (e.g., a vehicle 850, external device 810, exoskeleton server 830, admin device 840, or the like). For example, such data can include position and/or orientation data of the exoskeleton system 100 and/or a moving location where the exoskeleton system 100 is located (e.g., a ship, water vehicle, land vehicle, aircraft, or the like). The method can further include determining whether or not the user 101 is in danger of, is about to or is in the process of falling from a location where the exoskeleton system 100 is located (e.g., a ship, boat, truck, or the like). Where a determination is made that the user 101 is in danger of, is about to or is in the process of falling overboard from a location where the exoskeleton system 100 is located, a determination can be made (e.g., by the exoskeleton system 100, a computing device of a vehicle 850, an external device 810, an exoskeleton server 830, and admin device 840, or the like) regarding how to remediate the determined state of the user 101; and such a remediation can be performed by the exoskeleton system 100 and/or other systems such as a vehicle 850, an external device 810, an exoskeleton server 830, and admin device 840, or the like).

For example, such remediation can include generating an alert by the exoskeleton system 100 (e.g., audio, visual or haptic) for the user 101 or other persons or devices around the user 101; generating an alert at the location where the exoskeleton system 100 is present; actuating the exoskeleton system 100 to prevent falling overboard; actuating the exoskeleton system 100 to prepare for falling into water or on the ground; actuating the exoskeleton system 100 to assist the user 101 during falling into water or landing on the ground; actuating the exoskeleton system 100 to assist the user 101 after falling into water or landing on the ground; inflating one or more buoyancy cavities of the exoskeleton system 100; alert by the exoskeleton system 100 (e.g., audio, visual or haptic) so the user can be found by others after falling into water or landing on the ground; sending an alert to emergency services, and the like.

Additionally, forces, orientation, position, velocity, acceleration and the like measured by a vehicle 850 and shared with the exoskeleton system 100 and/or network 800 may be used by the exoskeleton system 100 and/or network 800 in some examples, in addition to sensor data from the exoskeleton system 100, to infer that the user 101 may have exited the vehicle 850. For example, a method of inferring that a user has exited a vehicle 850 can comprise obtaining sensor data from the vehicle 850 and obtaining sensor data from the exoskeleton system 100, with such sensor data corresponding to respective forces, orientation, position, velocity, acceleration, and the like, of the vehicle 850 and exoskeleton system 100.

The method can further include determining a correspondence between the sensor data of the vehicle 850 and exoskeleton system 100, and if a sufficient correspondence at a threshold level is determined, then a determination can be made that the user 101 and exoskeleton system 100 are riding in the vehicle 850. However, if there is not sufficient correspondence at a threshold level, then a determination can be made that the user 101 and exoskeleton system 100 are not riding in the vehicle 850 and/or that the user has exited or fallen from the vehicle 850.

For example, a user 101 wearing an exoskeleton system 100 can be walking towards an approaching vehicle 850 and a determination can be made that the user is not riding in the vehicle 850 based on the different forces, orientation, position, velocity, acceleration being experienced by the exoskeleton system 100 and vehicle 850. The user 101 can enter the vehicle 850, which can start driving to a destination and a determination can be made that the user is riding in the vehicle 850 based on similarity or correspondence of forces, orientation, position, velocity, acceleration being experienced by the exoskeleton system 100 and vehicle 850. The vehicle 850 can reach the destination and the user can exit the vehicle 850 and the user 101 can walk away from the vehicle 850 and the vehicle 850 may drive away. A determination can be made that the user 101 is not riding in the vehicle 850 based on the different forces, orientation, position, velocity, acceleration being experienced by the exoskeleton system 100 and vehicle 850.

In another embodiment, a user's exposure to chemicals, pollutants, and/or radiation can be measured and risk to the user can be inferred using sensors such as one or more Geiger counters, chemical sensors, infrared sensors, ultraviolet sensors, air pollutant sensors, time-of-flight sensors, laser depth sensors, and the like. Additionally, exposure can be inferred in some embodiments when an exoskeleton system 100 is cleaned and chemicals, pollutants, and/or radiation are measured on the exoskeleton system 100 or measured in a specific area of the exoskeleton system 100 or cleaning area that traps contaminants. This can be done in various examples as a regular procedure for professionals such as firefighters, first responders, scientists and the like who may use regular decontamination procedures.

For example, a method of determining a chemical, radiological or biological exposure can comprise obtaining data regarding chemical, radiological or biological exposure, which in some examples can include data from sensors discussed above. A determination can be made whether or not the data is indicative of an exposure such as presence of undesirable chemical, radiological or biological material(s) above a given threshold and/or for a given threshold time, and if so, in some embodiments, a determination can be made regarding a risk associated with such an exposure. For example, such a risk to a user can be based on a determined amount of the material(s) identified as being present, a length of time of exposure (e.g., based on exoskeleton system 100 use session data such as total time of use, determined time of when exposure begin, determined time of when exposure ended, changes in exposure amount, and the like). In various embodiments, if an exposure is determined, the exoskeleton system 100 can present an alert to the user 101 (e.g., to warn the user of the exposure, to alert the user to leave the area, or the like) and/or send an alert to another system (e.g., an external device 810, exoskeleton server 830, admin device 840, another exoskeleton system 100, or the like).

In another embodiment, the exoskeleton system 100 can use data to infer injuries sustained by the user 101. In one example embodiment, sensors can detect differences in the range of motion of a user's joint, such as by a rotary encoder on an actuator unit 110 measuring joint angles, while moving when compared to the user's normal baseline range of motion, where a diminished range of motion could indicate user injury. In other embodiments, movement indicative of injury such as a change in symmetrical gait, limping, and the like can be detected and inferred as a possible injury. In further embodiments of injury inference, sensors on the exoskeleton system 100 such as one or more ultrasound transducers, radio wave emitters and detectors, magnetic field emitters and detectors, EMG, and the like can be used to detect changes in physiological tissues such as damage to ligaments, tendons, muscles, bones, and the like.

In some embodiments, a method of determining an injury to a user 101 wearing an exoskeleton system 100 can include obtaining sensor data from an exoskeleton system 100 and determining whether the obtained sensor data is indicative of the user 101 having an injury. For example, a determination can be made that the data is indicative of the user having an asymmetrical gait, limping, or the like, which can result in determining an injury is present. In another example, a current movement profile or score can be generated based at least in part on the obtained data and the movement profile can be compared to a baseline movement profile for the specific user or for users generally. If differences in the profiles is indicative of an injury or the profile or score is below a given threshold, then a determination can be made that an injury is present. In response to determining that an injury is present, an alert can be generated and presented to the user 101 via the exoskeleton system 100 and/or an alert can be sent to an external device 810, exoskeleton server 830, admin device 840, or the like. In some embodiments, in response to determining that an injury is present, the configuration of the exoskeleton system 100 can be changed to support the identified injury during movement, one or more actuator units 110 can be stiffened to act as a splint or brace for the identified injury, or the like.

In one embodiment, data can be used to make inferences regarding the fatigue level of a user. In such an embodiment, some examples of sensors on the wearable robotic system that can collect data used to make such an inference can include but are not limited to one or more accelerometers, gyroscopes, magnetometers, sweat sensors, and the like. Using these types of sensors, some example measurements include but are not limited to trunk angular velocity and position in the roll/lateral and pitch/fore-aft planes, neck angle, step width, step length, step frequency, center of mass movement, walking speed, and changes in sweat level, any combination of which may be used as indicators of fatigue, including when compared to established baselines of such measurements taken when a user is uninjured or un-fatigued.

In some embodiments, a method of determining fatigue of a user 101 wearing an exoskeleton system 100 can including obtaining sensor data from an exoskeleton system 100 and determining whether the obtained sensor data is indicative of the user 101 being fatigued. For example, a determination can be made that the data is indicative of the user having an asymmetrical gait, limping, walking at slower pace, walking with a labored gait, walking with less control, or the like, which can result in determining fatigue is present. In another example, a current movement profile or score can be generated based at least in part on the obtained data and the movement profile can be compared to a baseline movement profile for the specific user or for users generally. If differences in the profiles is indicative of fatigue or the current profile or score is below a given threshold, then a determination can be made that fatigue is present. In some embodiments, a determination of fatigue can be based at least in part on a time of a current exoskeleton use session by the user; a time and/or times from one or more previous exoskeleton use sessions by the user; a calculated total exertion score for a current or previous exoskeleton use session by the user; a total number of steps walked; a user profile; and the like. For example, in some embodiments, a determination of fatigue can be weighted to be more likely to be determined based on increasing time of a current exoskeleton use session by the user; an increasing time and/or short time from one or more previous exoskeleton use sessions by the user; an increasing calculated total exertion score for a current or previous exoskeleton use session by the user; an increasing total number of steps walked; a user profile indicating typical use time(s), typical exertion, or typical use type(s); and the like.

In response to determining that fatigue is present, an alert can be generated and presented to the user 101 via the exoskeleton system 100 and/or an alert can be sent to an external device 810, exoskeleton server 830, admin device 840, or the like. In some embodiments, in response to determining that fatigue is present, the configuration of the exoskeleton system 100 can be changed to provide additional support for the user (e.g., increase force output by the actuator unit(s) 110, the user or other party can receive an alert suggesting that the user take a break, cease activity or cease activity within a determined amount of time.

In another embodiment, data from sensors of an exoskeleton system 100 can be used to make estimates of biomechanical metrics such as balance, step length, lateral step width, spatial gait symmetry, temporal gait symmetry, force-based gait symmetry, distance walked, elevation changes, walking speed, trunk angular velocity and position in the roll/lateral and pitch/fore-aft planes, step-to-step variability, step count based on terrain (e.g., level ground, incline/decline, upstairs/downstairs), angle of incline walked, duration of resting periods, and the like.

In a further embodiment, similar data can be used to infer the user's activities, such as walking, running, standing, sitting, stair climbing/descending, ramp climbing/descending, and the like. In one embodiment, data from the system's sensors can be used to make estimates of health metrics including, but not limited to, calories burned, resting heart rate, active heart rate, respiration rate, blood oxygen level, skin temperature, skin moisture, internal temperature, and the like. Sensors used to estimate health metrics may include, but are not limited to, one or more accelerometers, gyroscopes, thermistors, temperature sensors, pulse oximeters, time-of-flight sensors, depth sensors, humidity sensors, sweat sensors, pressure sensors, strain gauges, voltmeters, sonographic sensors, electrocardiograms, electroencephalograms, and the like.

In another embodiment, data from the sensors of an exoskeleton system 100 can be used to infer symptoms of illness in the user. In one example embodiment, it can be inferred that the user is coughing or has coughed by measuring the motions of the user. In this embodiment, sudden torso movements or muscle twitches can be indicative of coughing or vomiting and may be recorded by the system as coughs or vomiting by the user. In another embodiment, posture and time measurements may be used to detect if the user uses the bathroom, rests, sleeps, or the like. In another embodiment, the system may use one or more sweat sensors or temperature sensors to detect perspiration or abnormal skin temperature, which can also be inferred as a symptom of illness, fever, and/or physiological shock. Any one or combination of these inferences could be used to infer an illness and/or abnormal health state of the user.

In some embodiments, a method of determining illness of a user 101 wearing an exoskeleton system 100 can including obtaining sensor data from an exoskeleton system 100 and determining whether the obtained sensor data is indicative of the user 101 being ill. For example, a determination can be made that the data is indicative of the user having one of more symptoms of illness (e.g., as discussed above) which can result in determining illness is present. In another example, a current user health profile or score can be generated based at least in part on the obtained data and the movement profile can be compared to a baseline health profile or score for the specific user or for users generally. If differences in the profiles is indicative of illness or the current profile or score is below a given threshold, then a determination can be made that illness is present.

In response to determining that illness is present, an alert can be generated and presented to the user 101 via the exoskeleton system 100 and/or an alert can be sent to an external device 810, exoskeleton server 830, admin device 840, or the like. In some embodiments, in response to determining that illness is present, the configuration of the exoskeleton system 100 can be changed to provide additional support for the user (e.g., increase force output by the actuator unit(s) 110, the user or other party can receive an alert suggesting that the user take a break, cease activity or cease activity within a determined amount of time, a diagnosis of the illness, a suggestion to treat the illness (e.g., take pills or an injection) or the like.

In some example embodiments, the user can confirm inferred physical or other states (e.g., that they coughed or used the bathroom or took rest periods) on an external device 810 (e.g., on a smartphone through an application) that is operably connected to the exoskeleton system 100. This confirmation may be done in real time, at the end of the use period, and/or at the end of the day. In various embodiments, a method of obtaining user feedback on inferences can include making an inference regarding a state of the user 101, a state of the exoskeleton system 100, a state of a location where the user 101 and exoskeleton system 100 are present, or the like. The method can further include providing a prompt to the user 101 that presents the inference and/or that includes a feedback mechanism for the user to evaluate the inference. For example, an evaluation can be true/untrue, accurate/inaccurate, a sliding scale for the user to input a value within a range, a list of possible inferences that the user can select, or the like. In various embodiments, feedback from the user can be used (e.g., by the exoskeleton system 100, an exoskeleton server 830, an admin server 840, or the like) to improve or modify a classification program, an inference program, an operating program, or the like. For example, a user providing confirmation of inference accuracy can reinforce a system used to make such an inference or the user providing an indication of an incorrect inference can be used down-regulate or modify a system used to make such an inference.

Additionally, in various embodiments, the exoskeleton system 100 (or other device or system) may share the inferences of the user's physical states or changes in the user's physical states with the user or another entity, such as one or more other users, relevant parties, notification systems, care providers, other exoskeleton system 100, an external device 810, exoskeleton server 830, admin device 840, and the like. In some embodiments, inferences made by the exoskeleton system 100 (or other device or system) may be shared with the user or a third party to confirm accuracy. For example, a method of obtaining user feedback on inferences can include making an inference regarding a state of the user 101, a state of the exoskeleton system 100, a state of a location where the user 101 and exoskeleton system 100 are present, or the like. The method can further include providing a prompt to a third party (e.g., via an external device 810, an exoskeleton server 830 and admin device 840, or the like) that presents the inference and/or that includes a feedback mechanism for the user to evaluate the inference. Evaluations and use of evaluations can be as discussed above.

In other example embodiments, any combination of user health related inferences may be used to create a score grading the user's movement and/or health and/or the exoskeleton system's movement and/or health. In various embodiments, the exoskeleton system 100 may save data and/or such data can be saved on an external device 810, exoskeleton server 830, admin device 840, or the like, in order to track the data and related inferences being made over time and use the collected data and inferences to improve future inferences.

For example, a method of generating one or more health score of a user can include obtaining sensor data generated by or related to an exoskeleton system 100; associating the obtained sensor data with a user or exoskeleton system 100 (e.g., a user or exoskeleton ID or profile); generating a health score based at least in part on the obtained data; and storing the generated health score. Such a health score can be generated by the exoskeleton system 100, an external device 810, exoskeleton server 830, admin device 840, or the like, and can be stored at the system that generated the health score and/or communicated to another system. In some embodiments, a plurality of health scores generated can be associated with a user profile or an exoskeleton profile that can allow health of the user or exoskeleton system 100 to be tracked over time.

In various embodiments, the exoskeleton system 100 can respond to data and related inferences by changing the output of the exoskeleton system 100. In one example embodiment, the assistance level of the exoskeleton system 100 at the actuator unit(s) 110 may increase to support the user because a fall state, where the user is falling to the ground, is inferred or predicted above a threshold probability from the immediate data.

In some embodiments, data from multiple exoskeleton systems 100 (see e.g., FIG. 9) can be combined to make inferences about one or multiple exoskeleton systems 100. In one example embodiment, when multiple users 101 wearing a respective exoskeleton system 100 are in a vehicle 850 that crashes, the multiple exoskeleton system 100 could infer that a crash has occurred based on the accelerations measured by accelerometers of the exoskeleton system(s) 100. In a further embodiment, the direction and force of impact from the crash can be inferred from similar acceleration data collected by the exoskeleton system(s) 100.

For example, a plurality of users 101 wearing respective exoskeleton systems 100 (see, e.g., FIG. 9) can be walking towards an approaching vehicle 850 and a determination can be made that the users 101 are not riding together (e.g., in the vehicle 850) based on the different forces, orientation, position, velocity, acceleration being experienced by the respective exoskeleton systems 100. The users 101 can enter the vehicle 850, which can start driving to a destination and a determination can be made that the users 101 are riding in the vehicle 850 together based on similarity or correspondence of forces, orientation, position, velocity, acceleration being experienced by the respective exoskeleton systems 100. The vehicle 850 experiences a crash and a determination can be made that the users 101 riding in the vehicle 850 have been in a crash together based on similarity or correspondence of forces, orientation, position, velocity, acceleration being experienced by the respective exoskeleton systems 100 and a determination that such sensor data corresponds to or is indicative of a vehicle crash. In such embodiments, data from the vehicle 850 can be absent or unavailable or can also be used to make determinations regarding association of the users 101 with the vehicle and/or a vehicle crash.

In a further embodiment, the various users' level of consciousness could be inferred based on system motion after the crash, where lack of detectable motion would lead to an inference of user unconsciousness or immobility. For example, where a vehicle crash (or other harmful event such as a fall, explosion, exposure to smoke) is identified, lack of motion by an exoskeleton system 100 after the vehicle crash can be used to make a determination that the user 101 associated with exoskeleton system 100 is unconscious, deceased, or the like. Various embodiments can use the options listed above or combinations and variants thereof, but are in no way limited to the explicitly stated combinations of methods and items.

Sensor data and other relevant data can be used by the exoskeleton system 100 to make inferences about the cognitive states of a user wearing the exoskeleton system 100. These cognitive states can include, but are not limited to, the user's motor control ability, ability to complete complex tasks, cognitive fatigue, social interactions, stress levels, responsiveness, consciousness, and the like. In one embodiment, the exoskeleton system 100 uses data from sensors of the exoskeleton system 100 to analyze the user's ability to walk along a prescribed path such as a straight line, an outdoor trail, a beam, over a boulder field, through an environment with obstacles, through a minefield, and the like. In such an embodiment, the user's response to the prescribed path may allow the system to infer the motor control ability of the user and the ability to navigate terrains of various difficulties. In one embodiment, the exoskeleton system 100 can measure the user's ability to complete complex tasks. These tasks can be defined by various metrics, including but not limited to the number of actions to complete a task, the order of actions, the physical difficulty to complete each action, the cognitive difficulty to complete each action, and the effect of each action on another action, and the like. The exoskeleton system 100 in some examples can measure these task metrics with other related metrics such as, but not limited to, the time to complete a task metric, and number of motions to complete a task metric, and the like. The exoskeleton system 100 in some examples can also estimate the energy required to perform a task metric from measurements such as the amount of assistance received from the exoskeleton system 100, one or more EEG sensors, EKG sensors, EMG sensors, and the like. Measuring any combination of these metrics related to a user's ability to complete complex tasks can be used in various embodiments to infer the baseline cognitive load of the user, the cognitive fatigue of a user, and the like. In one example embodiment, measuring that a user took more time to complete a task metric than a previous baseline measurement could indicate that the user is cognitively fatigued. In another example embodiment, measuring that a user spent more energy or took more actions as measured by the wearable system than a previously measured baseline could indicate that a user was experiencing higher cognitive load than a baseline condition. In a further example embodiment, this baseline could be a baseline measurement taken previously of the user, a group of users, a calculated baseline based on a biomechanical simulation, and the like.

A method of testing user ability can comprise determining to initiate an ability test such as based on a schedule, an elapsed amount of use time, a determined state of the user, an indication by the user 101 (e.g., via an interface 515 of the exoskeleton device 510 and/or external device 810) an indication by a third party via an exoskeleton server 830, admin device 840, or the like. The method can further include providing an alert to the user 101 regarding the test, which can include instructions for performing one or more actions of a test (see e.g., discussed above). The user can perform the one or more actions of the test and sensor data generated during the test can be analyzed and a score or profile of the current ability can be generated, which in some examples can be based on a baseline ability score or profile of the user specifically or for users generally, previous ability score or profile of the user specifically or for users generally. For example, a baseline ability score or profile can be generated by having the user perform the one or more test actions one or more times to generate a baseline ability score or profile. A current ability score or profile can be stored at the exoskeleton system 100, external device 810, exoskeleton server 830, admin device 840, or the like and can be associated with user profile and/or exoskeleton profile. The current ability score or profile, baseline ability score or profile and/or previous ability scores or profiles can be used to make determinations regarding a state of the user 101, a state of the exoskeleton system 100 a state of a location where the user 101 and exoskeleton device are present, and the like (see e.g., discussed herein). Such a determination can be used in some examples to generate an alert for a user 101 or third party such as at the exoskeleton system 100, external device 810, exoskeleton server 830, admin device 840, or the like. For example, such an alert may indicate to the user to take a break, cease activity, seek medical attention, or the like.

In one embodiment, a user's social interactions can be measured and/or defined by the time spent within the vicinity of other people, a measurement of the proximity of the user to those same people, the frequency of meeting between the user and other people, and the like. These measurements related to people within the vicinity of the user can occur by the user's exoskeleton system 100 through sensors such as, but not limited to, distance sensors, LiDAR, radar, proximity sensors, and the like, or by the other people also wearing an exoskeleton system 100, whose presence is then communicated to each other, such as through a common wireless network 820 or to an exoskeleton server 830, admin device 840, or the like (see FIGS. 8 and 9).

A method of tracking social interaction of a user 101 wearing an exoskeleton system 100 can include identifying one or more persons and/or devices in proximity to the user 101 wearing an exoskeleton system 100 (see e.g., discussed above) and determining whether such one or more persons and/or devices should be counted as having a social interaction with the user 101 wearing an exoskeleton system 100. For example, such a determination can be based on determined proximity of the one or more persons and/or devices to the user 101 wearing an exoskeleton system 100; length of time of proximity of the one or more persons and/or devices to the user 101 wearing an exoskeleton system 100; orientation of the of the one or more persons and/or devices relative to the user 101 wearing an exoskeleton system 100 (e.g., are the persons facing each other, standing side by side, standing back-to-back, or the like. Positive and/or negative determinations of a social interaction can be stored at the exoskeleton system 100, external device 810, exoskeleton server 830, admin device 840, or the like. In some embodiments, positive and/or negative determinations of a social interaction can be stored associated with a determined identity of a person and/or device of the social interaction, a determined length of time of the social interaction, a determined type of the social interaction, determined characteristics of the social interaction, and the like.

In a further embodiment, when users of respective exoskeleton systems 100 are in close proximity, data can be collected on duration of meeting, if they were engaged in conversation, the frequency of meeting and conversing, and the like. In an example embodiment, audio sensors, such as microphones, as well as measurements of body kinematics and posture, gesticulation, head motion, and the like could be used to infer the nature, type or characteristics of the social interaction, including whether a user perceived the interaction as positive or negative based on the number of head nods in agreement, the volume and pitch of audible dialogue, presence of laughter, a slouched posture, and the like. For example, a method of determining nature, type or characteristics of a social interaction can include obtaining data from one or more exoskeleton systems 100; determining one or more persons associated with the social interaction; and determining nature, type or characteristics of a social interaction between the one or more persons based at least in part on the obtained data from the one or more exoskeleton systems 100.

In one embodiment, the exoskeleton system 100 infers the stress level of the user using sensors such as one or more accelerometers, gyroscopes, skin conductivity sensors, sweat sensors, skin temperature sensors, heart rate sensors, and the like. In an example embodiment, presence of high levels of perspiration combined with an elevated heart rate and tremors in the hands may indicate a high stress level of the user. For example, a method of determining a stress level of a user 101 wearing an exoskeleton system 100 can include obtaining data from the exoskeleton system 100; and determining a stress level of the user based at least in part on the obtained data. In some embodiments, such a stress level can be determined by and/or shared with one or more of the exoskeleton system 100, an external device 810, exoskeleton server 830, admin device 840, or the like. In some embodiments, a determined stress level can be used to generate an alert to the user 101 (e.g., via the exoskeleton system 100, an external device 810, or the like) or to a third party (e.g., via an exoskeleton server 830, admin device 840, another exoskeleton system 100, or the like). Such an alert an include a diagnosis of the stress, a suggestion to remediate the stress, a suggestion to seek assistance, a suggestion to assist the user, an indication of how to assist the user, or the like.

In another embodiment, the exoskeleton system 100 infers the responsiveness or alertness of a user. In an example embodiment, the exoskeleton system 100 may infer responsiveness using one or more accelerometers, gyroscopes, skin temperature sensors, heart rate sensors, EEG sensors, EMG sensors, and the like to detect if a user responds to an auditory noise from the exoskeleton system 100, an auditory noise from the environment, an actuation of the actuation device(s) 110 worn by the user, an external stimulus or stimulus provided by the exoskeleton system 100, and the like. In a further example embodiment, the exoskeleton system 100 can then assess the level of responsiveness by such metrics as measuring the time it takes the user to react, whether the user turns toward the sound, and the like. In one embodiment, this stimulus response data can be used to determine a user's state of consciousness, such as if they are asleep, unconscious, semi-conscious, conscious and the like. In an example embodiment, if no motion is detected, the exoskeleton system 100 may infer that the user is unconscious, or the exoskeleton system 100 may infer that the user is asleep if their motion pattern is similar to their sleep motion pattern.

A method of determining consciousness or responsiveness of a user 101 wearing an exoskeleton system 100 can include generating a stimulus such as a visual, auditory or haptic stimulus, which can be via the exoskeleton system 100, an external device 810 and/or another device. The method can further include obtaining data from the exoskeleton system 100 (and/or other device) at the time of and proximately after and/or before the generated stimulus; and determining a level of responsiveness to the stimulus, which can include lack of response, slow response, full response, or the like. In some examples, determining the level of responsiveness can be based at least in part on a baseline or previous responsiveness of the user to the stimulus or a baseline responsiveness for users generally.

Additional ways to infer unconsciousness may include, but are not limited to, a decrease in heart rate, a decrease in respiratory rate, a decrease in body temperature, decreased motion of the user, a combination of these measurements, and the like. In another embodiment, the exoskeleton system 100 can detect if a user is not breathing using sensors such as one or more accelerometers, gyroscopes, EMG sensors, and the like. In another embodiment, a user's presentation of high-altitude sickness may be inferred using sensors such as one or more accelerometers, gyroscopes, magnetometers, barometers, temperature sensors, heart rate sensors, pulse oximetry, and the like. These sensors may be used in some examples to measure symptoms such as respiratory distress, sleeping difficulty, vomiting, rapid breathing, loss of motor control, in the presence of altitudes greater than 6000 feet. Various embodiments can use the options listed above or combinations and variants thereof, but are in no way limited to the explicitly stated combinations of methods and items.

For example, a method of determining a consciousness level of a user 101 wearing an exoskeleton system 100 can include obtaining data from the exoskeleton system 100; and determining a consciousness level of the user based at least in part on the obtained data. In some embodiments, such a consciousness level can be determined by and/or shared with one or more of the exoskeleton system 100, an external device 810, exoskeleton server 830, admin device 840, or the like. In some embodiments, a determined consciousness level can be used to generate an alert to the user 101 (e.g., via the exoskeleton system 100, an external device 810, or the like) or to a third party (e.g., via an exoskeleton server 830, admin device 840, another exoskeleton system 100, or the like). Such an alert an include a diagnosis of the consciousness, a suggestion to remediate the consciousness level, a suggestion to seek assistance, a suggestion to assist the user, an indication of how to assist the user, an alert to emergency services, or the like.

As discussed herein, in various embodiments, the exoskeleton system 100 may save data locally at the exoskeleton system 100 (e.g., in the memory 512 of the exoskeleton device 510) and/or externally, such as uploaded to a cloud database, an external device 810, and exoskeleton server 630, an admin device 630, and the like, in order to track these states over time and use the data to improve future inferences about the states. Additionally, in various embodiments, the exoskeleton system 100 may share the user's data, whether collected from the exoskeleton system 100, another exoskeleton system 100, other related sensor systems, and the like, as well as inferences from that data, such as cognitive states or changes in the user's cognitive states, with the user or another entity, such as one or more other users, relevant parties, care providers, other wearable systems, notification systems, and the like (e.g., shared to an exoskeleton server 630, an admin device 630, or the like).

In one embodiment, inferences made about a user may be shared with the user, an administrator or a third party (e.g., shared to an exoskeleton server 630, an admin device 630, or the like) to confirm accuracy of the inference. In one example embodiment, this feedback loop, whether one-time, intermittent, continual, or some combination thereof, of inference-accuracy confirmation can be used to improve the ability to make and accuracy of the inferences by the exoskeleton system 100. For example, a method of obtaining user feedback on inferences and/or a method of obtaining feedback on inferences from one or more third party as discussed herein can be used to make inferences and improve accuracy of the inferences by the exoskeleton system 100.

Additionally, in various embodiments, the exoskeleton system 100 can use data and inferences to guide and inform changes to the configuration of the exoskeleton system 100. Some example embodiments include increasing the level of physical support to the user when fatigue has been inferred, moving the user's limb to elicit a response from the user when a sleep state has been inferred and sleeping is undesirable (e.g., while driving), alerting surrounding exoskeleton system 100 and their users that a particular user is in a high stress state inferred from an increased amount of perspiration and unsteady gait, and the like.

Additionally, in various embodiments, data from multiple exoskeleton systems 100 can be combined to make inferences. For example, a method of making an inference regarding the state of a user 101, a state of a first exoskeleton system 100 being worn by the user 101 a state of a location where the user 101 and first exoskeleton system 100 is present can include obtaining data from a plurality of exoskeleton systems 100, which may or may not include the first exoskeleton system 100 and making an inference based on the obtained data.

In addition to inferring one or more states of a user 101 wearing an exoskeleton system 100, in various embodiments the exoskeleton system 100 may infer states of itself, including but not limited to inferences such as damage to a component or subsystem, electronics and sensor failure or out-of-calibration state, low battery, and the like. In one embodiment, the system infers device failure by using sensors such as temperature sensors, pressure sensors, rotary and linear encoders, current sensors, accelerometers, gyroscopes, magnetometers, strain gauges, and the like. Some example embodiments may include, but are not limited to, using prolonged temperature exposure outside normal operating conditions to infer damage to one or more motors, sensors, device joints, device materials, and the like; using pressure sensors to measure a fluidic leak; using an encoder to measure if a joint angle is beyond its normal operating range or unable to sweep a normal operating range; using accelerometers, gyroscopes, magnetometers, strain gauges and the like to measure fracture in a component, and the like.

For example, a method of determining a state of an exoskeleton system 100 can include obtaining data from the exoskeleton system 100 and determining a state of the exoskeleton system 100 based at least in part on the obtained data. In some embodiments, such a state of the exoskeleton system 100 can be determined by and/or shared with one or more of the exoskeleton system 100, an external device 810, exoskeleton server 830, admin device 840, or the like. In some embodiments, a determined state of the exoskeleton system 100 can be used to generate an alert to the user 101 (e.g., via the exoskeleton system 100, an external device 810, or the like) or to a third party (e.g., via an exoskeleton server 830, admin device 840, another exoskeleton system 100, or the like). Such an alert an include an indication of the state of the exoskeleton system 100, a suggestion to remediate or repair the state of the exoskeleton system 100, a suggestion to seek assistance, a suggestion to assist the user, an indication of how to assist the user, an alert to emergency services, or the like.

In another embodiment, device usage metrics such as but not limited to usage frequency, joint range of motion distribution, usage cycles, distribution of assistance levels, impact forces, and the like may be used to infer how likely a device is to fail, when that failure may occur, what component or subassembly may fail, and the like. For example, a method of determining a time until failure of an exoskeleton system 100 or portion of an exoskeleton system can include determining a state of the exoskeleton system 100 (see e.g., discussed above), which can include a general state of the exoskeleton system 100 or specific components or portions thereof (e.g., sensors, actuators 130, cables 140, computing systems, fluidic systems, a battery, or the like). The method can further include determining a time until failure of the exoskeleton system 100 or specific components or portions of the exoskeleton system 100, which can be based on the current state, previous state or baseline state of the exoskeleton system 100 or specific components or portions; based on a profile of the exoskeleton system 100 or specific components or portions; or the like.

As discussed herein, in various embodiments, the exoskeleton system 100 may save data locally at the exoskeleton system 100 (e.g., in the memory 512 of the exoskeleton device 510) and/or externally, such as uploaded to a cloud database, an external device 810, and exoskeleton server 630, an admin device 630, and the like, in order to track these states over time and use the data to improve future inferences about the states. Additionally, in various embodiments, the exoskeleton system 100 may share the user's data, whether collected from the exoskeleton system 100, another exoskeleton system 100, other related sensor systems, and the like, as well as inferences from that data, such as inferences regarding one or more states of the exoskeleton system 100, with the user or another entity, such as one or more other users, relevant parties, care providers, other wearable systems, notification systems, and the like (e.g., shared to an exoskeleton server 630, an admin device 630, or the like).

In one embodiment, inferences made about a user may be shared with the user, an administrator or a third party (e.g., shared to an exoskeleton server 630, an admin device 630, or the like) to confirm accuracy of the inference. In one example embodiment, this feedback loop, whether one-time, intermittent, continual, or some combination thereof, of inference-accuracy confirmation can be used to improve the ability to make and accuracy of the inferences made by the exoskeleton system 100 regarding one or more states of the exoskeleton system 100.

Additionally, in various embodiments, the exoskeleton system 100 can use data and inferences to guide and inform changes to the configuration of the exoskeleton system 100. Some example embodiments include a notification being sent to the user when a failure is detected or inferred in the system; decreasing an assistance level because a failure is detected or inferred that limits the ability of the exoskeleton system 100 to provide assistance; increasing the assistance level to overcome a detected or inferred failure such as a small leak in a fluidic actuator 130 whereby increasing the flow rate and/or pressure of fluid into the actuator could help compensate for the leak; deactivating the exoskeleton system 100 when a critical failure (such as pressure release valve no longer being operable which could lead to a dangerous overpressure situation), is detected or inferred that could endanger the user 101 or exoskeleton system 100 if operation continued; and the like.

In some embodiments, data from multiple exoskeleton system 100 (see e.g., FIG. 9) can be combined to make inferences about one or multiple systems. For example, a method of making an inference regarding the state of a user 101, a state of a first exoskeleton system 100 being worn by the user 101, a state of a location where the user 101 and first exoskeleton system 100 is present can include obtaining data from a plurality of exoskeleton systems 100, which may or may not include the first exoskeleton system 100 and making an inference based on the obtained data.

In addition to inferring states about the user 101 and itself, the exoskeleton system 100 in some examples may make inferences about the surrounding environment, such as but not limited to the weather, terrain, obstacles, and the like. In one embodiment, the exoskeleton system 100 is able to detect or infer its location on Earth. In one example embodiment, the exoskeleton system 100 may use an integrated GPS sensor to collect location information or may use the GPS sensor of a nearby device such as a smartphone (e.g., external device 810). In a further example embodiment, the location information of the user may be used to inform the user or relevant third parties about unsafe weather and/or terrain conditions such as an avalanche zone, a flash flood area, an earthquake zone, extreme low or high temperatures, and the like.

In another further example embodiment, location information may also be used by the exoskeleton system 100 to determine locations frequented and paths traversed by a user or any group of users of the exoskeleton system 100. For example, a method of path identification can include obtaining current location data of an exoskeleton system 100 (e.g., GPS data); determining a current location of the exoskeleton system 100 based at least in part on the current location data; and obtaining path data proximate the current location. In various embodiments, such path data can be presented to the user 101 (e.g., via an interface 515, external device 810, or the like), which can include routing instructions, and the like. In various embodiments, path data can be generated based on previous use session of the exoskeleton system 100 and/or other exoskeleton systems 100. In some embodiments, such path data can be stored at and obtained from the exoskeleton system 100, external device 810, exoskeleton server 830, admin device 840, or the like.

In one embodiment, the exoskeleton system 100 may collect information on environmental conditions. In some example embodiments, these environmental conditions can include, but are not limited to, visibility, humidity, temperature, wind speed and the like, and may be measured by sensors that include but are not limited to one or more lidar sensors, time-of-flight sensors, laser depth sensors, humidity sensors, temperature sensors, anemometers and the like. In an example embodiment, the exoskeleton system 100 can use sensors such as one or more barometers, anemometers, humidity sensors, temperature sensors, and the like to infer future changes in weather conditions such as storms, rain, and the like.

In one embodiment, the exoskeleton system 100 can make inferences about the surrounding terrain. In an example embodiment, the exoskeleton system 100 can use one or more barometers to measure the elevation of terrain. In another example embodiment, the exoskeleton system 100 uses sensors such as one or more GPS sensors, accelerometers, gyroscopes, magnetometers, barometers and the like to determine inclination of the terrain. In a further example embodiment, the exoskeleton system 100 may use elevation, inclination, weather conditions, health metrics, inferred fatigue level, and the like to infer the difficulty level of the terrain. In one embodiment, an exoskeleton system 100 can infer that it is in a dangerous environment, such as but not limited to low visibility from dust, night time, fog, snow, and the like, being buried from an avalanche, rockslide, snow, and the like, submersion in water, liquid, slurry, and the like, and other similar dangerous situations.

In some embodiments, a method of determining a state of a location where an exoskeleton system 100 is can include obtaining data from the exoskeleton system 100 (see e.g., examples above) and determining a state of a location where an exoskeleton system 100 is based at least in part on the obtained data. In some embodiments, such a state of the exoskeleton system 100 can be determined by and/or shared with one or more of the exoskeleton system 100, an external device 810, exoskeleton server 830, admin device 840, or the like. In some embodiments, a determined state of a location where an exoskeleton system 100 can be used to generate an alert to the user 101 (e.g., via the exoskeleton system 100, an external device 810, or the like) or to a third party (e.g., via an exoskeleton server 830, admin device 840, another exoskeleton system 100, or the like). Such an alert can include an indication of the state of the location where an exoskeleton system 100 is located, a suggestion to remediate the state of the location where an exoskeleton system 100 is located, a suggestion to seek shelter, a weather forecast, a route or alternative route, or the like.

In one example embodiment, low visibility environments can be detected by sensors such as but not limited to light sensors, infrared sensors, hygrometers for humidity, temperature sensors, and the like. In another example embodiment, being buried can be detected by sensors such as but not limited to pressure sensors detecting the pressure of material such as snow, rocks, gravel, debris and the like on the system, light sensors and a timekeeper detecting the absence of sunlight, audio sensors that detected a rockslide or avalanche event, and the like. In a further example embodiment, in response to inferring a dangerous environment, the exoskeleton system 100 can make itself easier to find for a user, other relevant parties, other systems and the like by emitting audio noise, emitting radio signals over multiple bandwidths, emitting light signals, actuating the system, activating a GPS or other location beacon, increasing system temperature, increasing pressure in the actuators, and the like.

In various embodiments, operating an exoskeleton system 100 based on visibility conditions can include obtaining visibility data (e.g., sensor data as discussed above); determining a visibility level; and where the visibility level is below a given threshold, determining a low-visibility state; determining a low-visibility response; and generating the determined low-visibility response. In some embodiments, determining a low visibility state and/or determining the low-visibility response can be based on a determined cause of the visibility level, a time that the visibility level has been present, a time of day (e.g., daytime, nighttime, dusk, dawn), weather or environment conditions, a mission or task profile, and the like. In various embodiments a low-visibility response can include an alert provided to the user (e.g., via an interface 515, external device 810, or the like) such as a suggestion to turn on a light, a routing or re-routing indication, an alert discussed above, or the like. In various embodiments a low-visibility response can include automatically turning on a light, a response as discussed above, or the like.

In one embodiment, ground mechanical properties such as density, hardness, mechanical impedance, and the like can be inferred by the exoskeleton system 100 using sensors such as one or more strain gauges, accelerometers, gyroscopes, magnetometers, rotary and linear encoders, sonographic sensors, ultrasound sensors, radio signal emitters and receivers, and the like. In one example embodiment, when more unstable terrain such as sand or mud are inferred by sensing a reduction in density and hardness of the ground, the exoskeleton system 100 can change its assistance level to compensate for the added difficulty of the user to traverse such a terrain.

In various embodiments, a method of operating an exoskeleton system 100 based on determined terrain can include obtaining sensor data (see e.g., examples above); determining one or more terrain characteristics based on the sensor data; determining a response based at least in part on the determined one or more terrain characteristics; and generating the response. For example, determining such a response can be based on a determined terrain type, a determined health or fatigue level of the user, a weather forecast, or the like.

In another embodiment, the exoskeleton system 100 may measure air quality of the environment using previously mentioned sensors to measure an amount of pollutants, particulate matter, chemical contaminants, radioactive contaminants, biological contaminant, and the like in the air or environment. In another embodiment, the exoskeleton system 100 can use sensors such as one or more laser depth sensors, radio signal emitters and receivers, lidar sensors, time-of-flight sensors, accelerometers, gyroscopes, magnetometers, and the like to map interior layouts of buildings such as size, type, rooms, layout, personnel, and the like.

As discussed herein, in various embodiments, the exoskeleton system 100 may save data locally at the exoskeleton system 100 (e.g., in the memory 512 of the exoskeleton device 510) and/or externally, such as uploaded to a cloud database, an external device 810, and exoskeleton server 630, an admin device 630, and the like, in order to track these states over time and use the data to improve future inferences about the state(s) of an environment where the exoskeleton system 100 is located. Additionally, in various embodiments, the exoskeleton system 100 may share the user's data, whether collected from the exoskeleton system 100, another exoskeleton system 100, other related sensor systems, and the like, as well as inferences from that data, such as inferences about the state(s) of an environment where the exoskeleton system 100 is located, with the user or another entity, such as one or more other users, relevant parties, care providers, other wearable systems, notification systems, and the like (e.g., shared to an exoskeleton server 630, an admin device 630, or the like).

In one embodiment, inferences made about a user may be shared with the user, an administrator or a third party (e.g., shared to an exoskeleton server 630, an admin device 630, or the like) to confirm accuracy of the inference. In one example embodiment, this feedback loop, whether one-time, intermittent, continual, or some combination thereof, of inference-accuracy confirmation can be used to improve the ability to make and accuracy of the inferences by the exoskeleton system 100 regarding an environment where the exoskeleton system 100 is present.

Additionally, in various embodiments, the exoskeleton system 100 can use data and inferences to guide and inform changes to the configuration of the exoskeleton system 100. Some example embodiments include a notification being sent to the user or other users when it is inferred that the user is entering a dangerous environment or inclement weather; increasing the assistance level when the terrain is inferred as difficult; deactivating the exoskeleton system 100 when the presence of lightning is inferred; and the like.

In various embodiments, data from multiples exoskeleton systems 100 can be combined to make inferences about the environment. For example, a method of making an inference regarding the state of a user 101, a state of a first exoskeleton system 100 being worn by the user 101 a state of a location where the user 101 and first exoskeleton system 100 is present can include obtaining data from a plurality of exoskeleton systems 100, which may or may not include the first exoskeleton system 100 and making an inference based on the obtained data. Such inferences can be used to provide alerts to one or more of the exoskeleton systems 100, to change the configuration of one or more of the exoskeleton systems 100, to generate further inferences by the one or more of the exoskeleton systems 100, and the like.

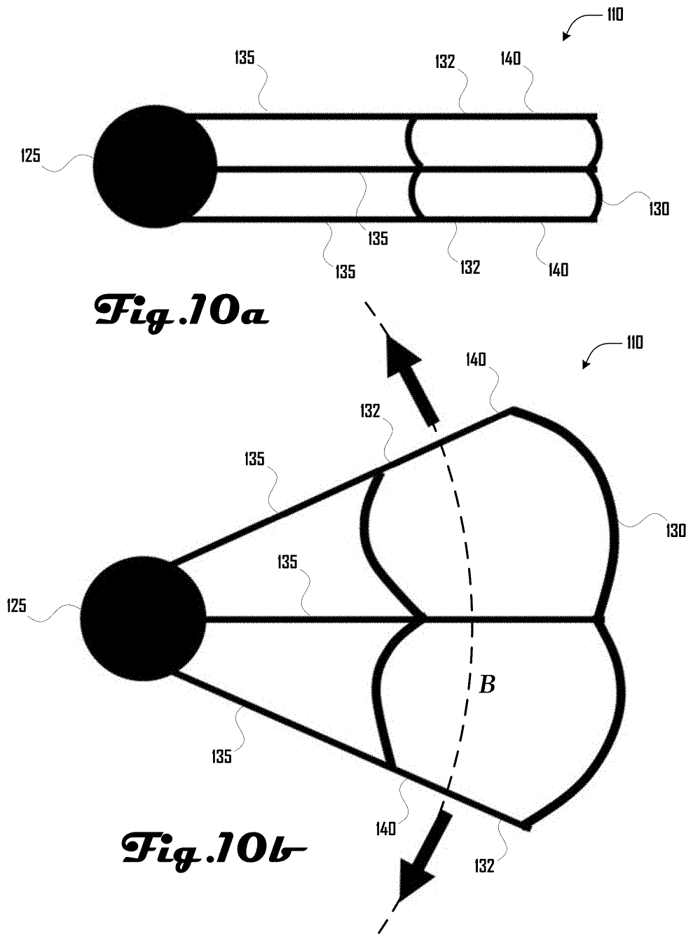

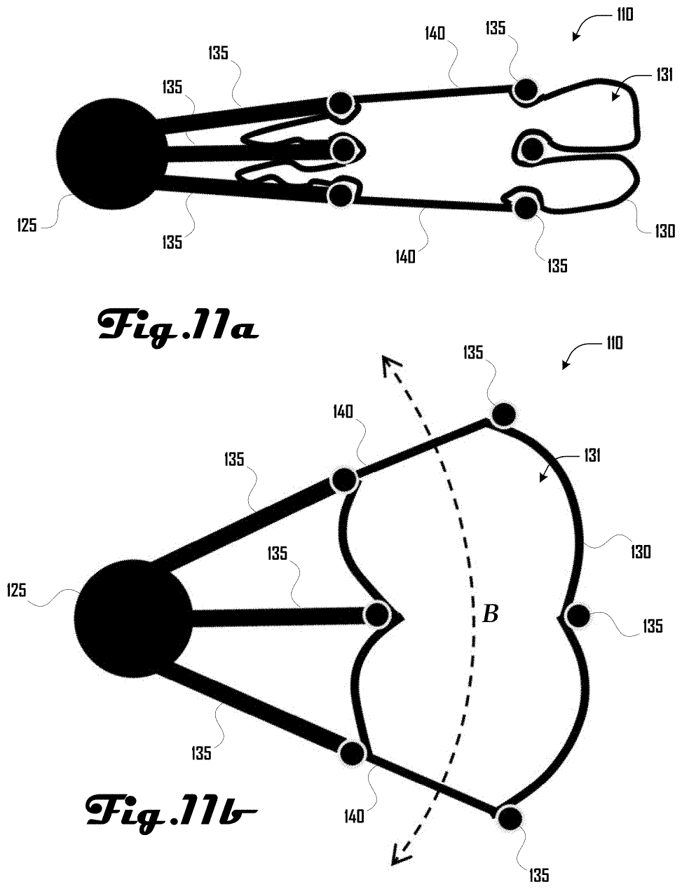

Turning to FIGS. 10a, 10b, 11a and 11b, examples of a leg actuator unit 110 can include the joint 125, bellows actuator 130, constraint ribs 135, and base plates 140. More specifically, FIG. 10a illustrates a side view of a leg actuator unit 110 in a compressed configuration and FIG. 10b illustrates a side view of the leg actuator unit 110 of FIG. 10a in an expanded configuration. FIG. 11a illustrates a cross-sectional side view of a leg actuator unit 110 in a compressed configuration and FIG. 11b illustrates a cross-sectional side view of the leg actuator unit 110 of FIG. 11a in an expanded configuration.

As shown in FIGS. 10a, 10b, 11a and 11b, the joint 125 can have a plurality of constraint ribs 135 extending from and coupled to the joint 125, which surround or abut a portion of the bellows actuator 130. For example, in some embodiments, constraint ribs 135 can abut the ends 132 of the bellows actuator 130 and can define some or all of the base plates 140 that the ends 132 of the bellows actuator 130 can push against. However, in some examples, the base plates 140 can be separate and/or different elements than the constraint ribs 135 (e.g., as shown in FIG. 1). Additionally, one or more constraint ribs 135 can be disposed between ends 132 of the bellows actuator 130. For example, FIGS. 10a, 10b, 11a and 11b illustrate one constraint rib 135 disposed between ends 132 of the bellows actuator 130; however, further embodiments can include any suitable number of constraint ribs 135 disposed between ends of the bellows actuator 130, including 1, 2, 3, 4, 5, 6, 7, 8, 9, 10, 15, 20, 25, 30, 50, 100 and the like. In some embodiments, constraint ribs can be absent.

As shown in cross sections of FIGS. 11a and 11b, the bellows actuator 130 can define a cavity 131 that can be filled with fluid (e.g., air), to expand the bellows actuator 130, which can cause the bellows to elongate along axis B as shown in FIGS. 10b and 11b. For example, increasing a pressure and/or volume of fluid in the bellows actuator 130 shown in FIG. 10a can cause the bellows actuator 130 to expand to the configuration shown in FIG. 10b. Similarly, increasing a pressure and/or volume of fluid in the bellows actuator 130 shown in FIG. 11a can cause the bellows actuator 130 to expand to the configuration shown in FIG. 11b. For clarity, the use of the term “bellows” is to describe a component in the described actuator unit 110 and is not intended to limit the geometry of the component. The bellows actuator 130 can be constructed with a variety of geometries including but not limited to a constant cylindrical tube, a cylinder of varying cross-sectional area, a 3-D woven geometry that inflates to a defined arc shape, and the like. The term “bellows” should not be construed to necessarily include a structure having convolutions.

Alternatively, decreasing a pressure and/or volume of fluid in the bellows actuator 130 shown in FIG. 10b can cause the bellows actuator 130 to contract to the configuration shown in FIG. 10a. Similarly, decreasing a pressure and/or volume of fluid in the bellows actuator 130 shown in FIG. 11b can cause the bellows actuator 130 to contract to the configuration shown in FIG. 11a. Such increasing or decreasing of a pressure or volume of fluid in the bellows actuator 130 can be performed by pneumatic system 520 and cables 145 of the exoskeleton system 100, which can be controlled by the exoskeleton device 510 (see FIG. 5).

In one preferred embodiment, the bellows actuator 130 can be inflated with air; however, in further embodiments, any suitable fluid can be used to inflate the bellows actuator 130. For example, gasses including oxygen, helium, nitrogen, and/or argon, or the like can be used to inflate and/or deflate the bellows actuator 130. In further embodiments, a liquid such as water, an oil, or the like can be used to inflate the bellows actuator 130. Additionally, while some examples discussed herein relate to introducing and removing fluid from a bellows actuator 130 to change the pressure within the bellows actuator 130, further examples can include heating and/or cooling a fluid to modify a pressure within the bellows actuator 130.

As shown in FIGS. 10a, 10b, 11a and 11b, the constraint ribs 135 can support and constrain the bellows actuator 130. For example, inflating the bellows actuator 130 causes the bellows actuator 130 to expand along a length of the bellows actuator 130 and can also cause the bellows actuator 130 to expand radially. The constraint ribs 135 can constrain radial expansion of a portion of the bellows actuator 130. Additionally, as discussed herein, the bellows actuator 130 can comprise a material that is flexible in one or more directions and the constraint ribs 135 can control the direction of linear expansion of the bellows actuator 130. For example, in some embodiments, without constraint ribs 135 or other constraint structures the bellows actuator 130 would herniate or bend out of axis uncontrollably such that suitable force would not be applied to the base plates 140 such that the arms 115, 120 would not be suitably or controllably actuated. Accordingly, in various embodiments, the constraint ribs 135 can be desirable to generate a consistent and controllable axis of expansion B for the bellows actuator 130 as they are inflated and/or deflated.

In some examples, the bellows actuator 130 in a deflated configuration can substantially extend past a radial edge of the constraint ribs 135 and can retract during inflation to extend less past the radial edge of the constraint ribs 135, to extend to the radial edge of the constraint ribs 135, or not to extend less past the radial edge of the constraint ribs 135. For example, FIG. 11a illustrates a compressed configuration of the bellows actuator 130 where the bellows actuator 130 substantially extends past a radial edge of the constraint ribs 135 and FIG. 11b illustrates the bellows actuator 130 retracting during inflation to extend less past the radial edge of the constraint ribs 135 in an inflated configuration of the bellows actuator 130.

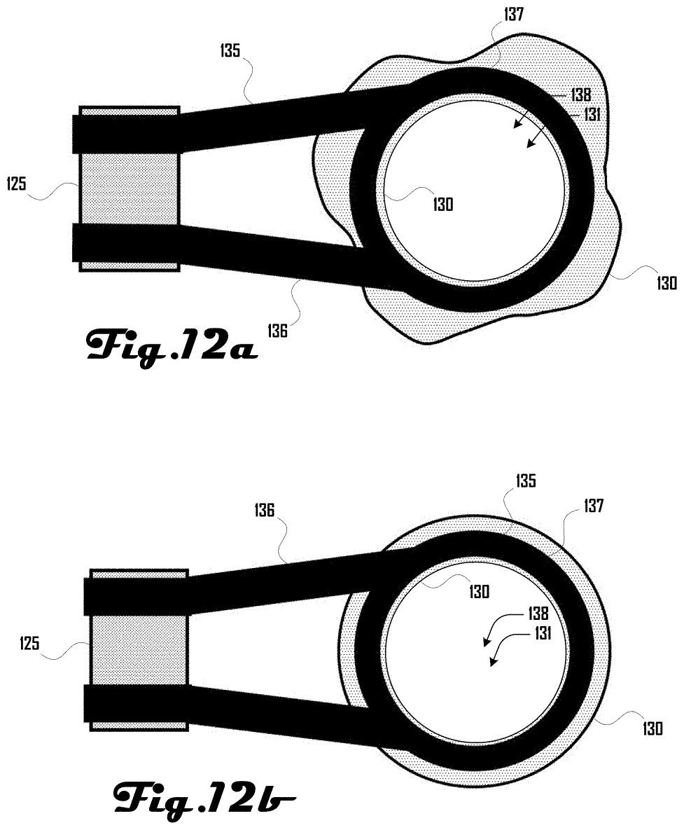

Similarly, FIG. 12a illustrates a top view of a compressed configuration of bellows actuator 130 where the bellows actuator 130 substantially extends past a radial edge of constraint ribs 135 and FIG. 12b illustrates a top view where the bellows actuator 130 retracts during inflation to extend less past the radial edge of the constraint ribs 135 in an inflated configuration of the bellows actuator 130.

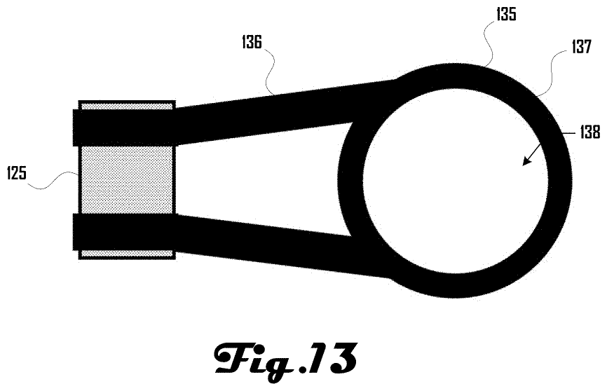

Constraint ribs 135 can be configured in various suitable ways. For example, FIGS. 12a, 12b and 13 illustrate a top view of an example embodiment of a constraint rib 135 having a pair of rib arms 136 that extend from the joint structure 125 and couple with a circular rib ring 137 that defines a rib cavity 138 through which a portion of the bellows actuator 130 can extend (e.g., as shown in FIGS. 11a, 11b, 12a and 12b). In various examples, the one or more constraint ribs 135 can be a substantially planar element with the rib arms 136 and rib ring 137 being disposed within a common plane.

In further embodiments, the one or more constraint ribs 135 can have any other suitable configuration. For example, some embodiments can have any suitable number of rib arms 136, including one, two, three, four, five, or the like. Additionally, the rib ring 137 can have various suitable shapes and need not be circular, including one or both of an inner edge that defines the rib cavity 138 or an outer edge of the rib ring 137.

In various embodiments, the constraining ribs 135 can be configured to direct the motion of the bellows actuator 130 through a swept path about some instantaneous center (which may or may not be fixed in space) and/or to prevent motion of the bellows actuator 130 in undesired directions, such as out-of-plane buckling. As a result, the number of constraining ribs 135 included in some embodiments can vary depending on the specific geometry and loading of the leg actuator unit 110. Examples can range from one constraining rib 135 up to any suitable number of constraining ribs 135; accordingly, the number of constraining ribs 135 should not be taken to limit the applicability of the invention. Additionally, constraining ribs 135 can be absent in some embodiments.

The one or more constraining ribs 135 can be constructed in a variety of ways. For example, the one or more constraining ribs 135 can vary in construction on a given leg actuator unit 110, and/or may or may not require attachment to the joint structure 125. In various embodiments, the constraining ribs 135 can be constructed as an integral component of a central rotary joint structure 125. An example embodiment of such a structure can include a mechanical rotary pin joint, where the constraining ribs 135 are connected to and can pivot about the joint 125 at one end of the joint structure 125, and are attached to an inextensible outer layer of the bellows actuator 130 at the other end. In another set of embodiments, the constraining ribs 135 can be constructed in the form of a single flexural structure that directs the motion of the bellows actuator 130 throughout the range of motion for the leg actuator unit 110. Another example embodiment uses a flexural constraining rib 135 that is not connected integrally to the joint structure 125 but is instead attached externally to a previously assembled joint structure 125. Another example embodiment can comprise the constraint ribs 135 being composed of pieces of fabric wrapped around the bellows actuator 130 and attached to the joint structure 125, acting like a hammock to restrict and/or guide the motion of the bellows actuator 130. There are additional methods available for constructing the constraining ribs 135 that can be used in additional embodiments that include but are not limited to a linkage, a rotational flexure connected around the joint structure 125, and the like.

In some examples, a design consideration for constraining ribs 135 can be how the one or more constraining ribs 135 interact with the bellows actuator 130 to guide the path of the bellows actuator 130. In various embodiments, the constraining ribs 135 can be fixed to the bellows actuator 130 at predefined locations along the length of the bellows actuator 130. One or more constraining ribs 135 can be coupled to the bellows actuator 130 in various suitable ways, including but not limited to sewing, mechanical clamps, geometric interference, direct integration, and the like. In other embodiments, the constraining ribs 135 can be configured such that the constraining ribs 135 float along the length of the bellows actuator 130 and are not fixed to the bellows actuator 130 at predetermined connection points. In some embodiments, the constraining ribs 135 can be configured to restrict a cross sectional area of the bellows actuator 130. An example embodiment can include a tubular bellows actuator 130 attached to a constraining rib 135 that has an oval cross section, which in some examples can be a configuration to reduce the width of the bellows actuator 130 at that location when the bellows actuator 130 is inflated.

The bellows actuator 130 can have various functions in some embodiments, including containing operating fluid of the leg actuator unit 110, resisting forces associated with operating pressure of the leg actuator unit 110, and the like. In various examples, the leg actuator unit 110 can operate at a fluid pressure above, below or at about ambient pressure. In various embodiments, the bellows actuator 130 can comprise one or more flexible, yet inextensible or practically inextensible materials in order to resist expansion (e.g., beyond what is desired in directions other than an intended direction of force application or motion) of the bellows actuator 130 beyond what is desired when pressurized above ambient pressure. Additionally, the bellows actuator 130 can comprise an impermeable or semi-impermeable material in order to contain the actuator fluid.

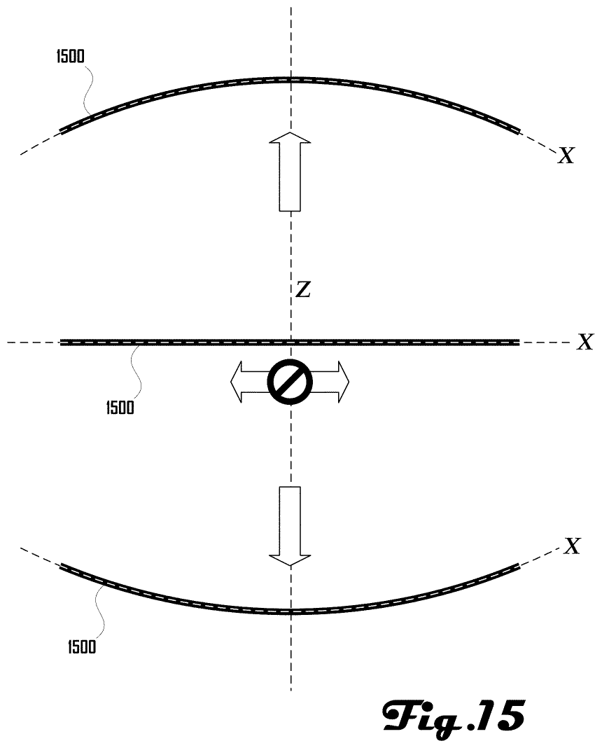

For example, in some embodiments, the bellows actuator 130 can comprise a flexible sheet material such as woven nylon, rubber, polychloroprene, a plastic, latex, a fabric, or the like. Accordingly, in some embodiments, the bellows actuator 130 can be made of a planar material that is substantially inextensible along one or more plane axes of the planar material while being flexible in other directions. For example, FIG. 15 illustrates a side view of a planar material 1500 (e.g., a fabric) that is substantially inextensible along axis X that is coincident with the plane of the material 1500, yet flexible in other directions, including axis Z. In the example of FIG. 15, the material 1500 is shown flexing upward and downward along axis Z while being inextensible along axis X. In various embodiments, the material 1500 can also be inextensible along an axis Y (not shown) that is also coincident with the plane of the material 1500 like axis X and perpendicular to axis X.

In some embodiments, the bellows actuator 130 can be made of a non-planar woven material that is inextensible along one or more axes of the material. For example, in one embodiment the bellows actuator 130 can comprise a woven fabric tube. Woven fabric material can provide inextensibility along the length of the bellows actuator 130 and in the circumferential direction. Such embodiments can still be able to be configured along the body of the user 101 to align with the axis of a desired joint on the body 101 (e.g., the knee 103).

In various embodiments, the bellows actuator 130 can develop its resulting force by using a constrained internal surface length and/or external surface length that are a constrained distance away from each other (e.g., due to an inextensible material as discussed above). In some examples, such a design can allow the bellows actuator 130 to contract, but when pressurized to a certain threshold, the bellows actuator 130 can direct the forces axially by pressing on the plates 140 of the leg actuator unit 110 because there is no ability for the bellows actuator 130 to expand further in volume otherwise due to being unable to extend its length past a maximum length defined by the body of the bellows actuator 130.

In other words, the bellows actuator 130 can comprise a substantially inextensible textile envelope that defines a chamber that is made fluid-impermeable by a fluid-impermeable bladder contained in the substantially inextensible textile envelope and/or a fluid-impermeable structure incorporated into the substantially inextensible textile envelope. The substantially inextensible textile envelope can have a predetermined geometry and a non-linear equilibrium state at a displacement that provides a mechanical stop upon pressurization of the chamber to prevent excessive displacement of the substantially inextensible textile actuator.

In some embodiments, the bellows actuator 130 can include an envelope that consists or consists essentially of inextensible textiles (e.g., inextensible knits, woven, non-woven, etc.) that can prescribe various suitable movements as discussed herein. Inextensible textile bellows actuator 130 can be designed with specific equilibrium states (e.g., end states or shapes where they are stable despite increasing pressure), pressure/stiffness ratios, and motion paths. Inextensible textile bellows actuator 130 in some examples can be configured accurately delivering high forces because inextensible materials can allow greater control over directionality of the forces.

Accordingly, some embodiments of inextensible textile bellows actuator 130 can have a pre-determined geometry that produces displacement mostly via a change in the geometry between the uninflated shape and the pre-determined geometry of its equilibrium state (e.g., fully inflated shape) due to displacement of the textile envelope rather than via stretching of the textile envelope during a relative increase in pressure inside the chamber; in various embodiments, this can be achieved by using inextensible materials in the construction of the envelope of the bellows actuator 130. As discussed herein, in some examples “inextensible” or “substantially inextensible” can be defined as expansion by no more than 10%, no more than 5%, or no more than 1% in one or more direction.

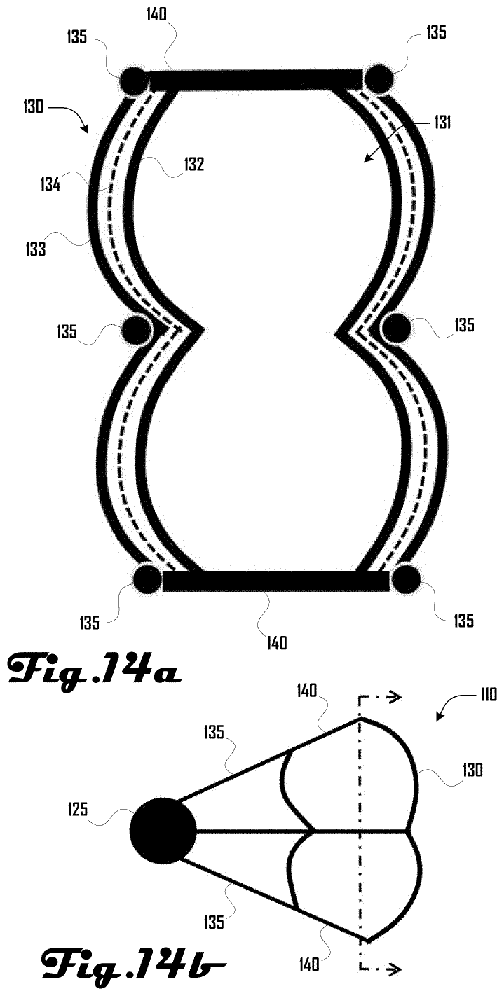

FIG. 14a illustrates a cross-sectional view of a pneumatic actuator unit 110 including bellows actuator 130 in accordance with another embodiment and FIG. 14b illustrates a side view of the pneumatic actuator unit 110 of FIG. 14a in an expanded configuration showing the cross section of FIG. 14a. As shown in FIG. 14a, the bellows actuator 130 can comprise an internal first layer 132 that defines the bellows cavity 131 and can comprise an outer second layer 133 with a third layer 134 disposed between the first and second layers 132, 133. Throughout this description, the use of the term “layer” to describe the construction of the bellows actuator 130 should not be viewed as limiting to the design. The use of ‘layer’ can refer to a variety of designs including a planar material sheet, a wet film, a dry film, a rubberized coating, a co-molded structure, and the like.

In some examples, the internal first layer 132 can comprise a material that is impermeable or semi-permeable to the actuator fluid (e.g., air) and the external second layer 133 can comprise an inextensible material as discussed herein. For example, as discussed herein, an impermeable layer can refer to an impermeable or semi-permeable layer and an inextensible layer can refer to an inextensible or a practically inextensible layer.

In some embodiments comprising two or more layers, the internal layer 132 can be slightly oversized compared to an inextensible outer second layer 133 such that the internal forces can be transferred to the high-strength inextensible outer second layer 133. One embodiment comprises a bellows actuator 130 with an impermeable polyurethane polymer film inner first layer 132 and a woven nylon braid as the outer second layer 133.

The bellows actuator 130 can be constructed in various suitable ways in further embodiments, which can include a single-layer design that is constructed of a material that provides both fluid impermeability and that is sufficiently inextensible. Other examples can include a complex bellows assembly that comprises multiple laminated layers that are fixed together into a single structure. In some examples, it can be necessary to limit the deflated stack height of the bellows actuator 130 to maximize the range of motion of the leg actuator unit 110. In such an example, it can be desirable to select a low-thickness fabric that meets the other performance needs of the bellows actuator 130.

In yet another embodiment, it can be desirable to reduce friction between the various layers of the bellows actuator 130. In one embodiment, this can include the integration of a third layer 134 that acts as an anti-abrasive and/or low friction intermediate layer between the first and second layers 132, 133. Other embodiments can reduce the friction between the first and second layers 132, 133 in alternative or additional ways, including but not limited to the use of a wet lubricant, a dry lubricant, or multiple layers of low friction material. Accordingly, while the example of FIG. 12a illustrates an example of a bellows actuator 130 comprising three layers 132, 133, 134, further embodiments can include a bellows actuator 130 having any suitable number of layers, including one, two, three, four, five, ten, fifteen, twenty-five, and the like. Such one or more layers can be coupled along adjoining faces in part or in whole, with some examples defining one or more cavities between layers. In such examples, material such as lubricants or other suitable fluids can be disposed in such cavities, or such cavities can be effectively empty. Additionally, as described herein, one or more layers (e.g., the third layer 134) need not be a sheet or planar material layer as shown in some examples and can instead comprise a layer defined by a fluid. For example, in some embodiments, the third layer 134 can be defined by a wet lubricant, a dry lubricant, or the like.

The inflated shape of the bellows actuator 130 can be important to the operation of the bellows actuator 130 and/or leg actuator unit 110 in some embodiments. For example, the inflated shape of the bellows actuator 130 can be affected through the design of both an impermeable and inextensible portion of the bellows actuator 130 (e.g., the first and second layer 132, 133). In various embodiments, it can be desirable to construct one or more of the layers 132, 133, 134 of the bellows actuator 130 out of various two-dimensional panels that may not be intuitive in a deflated configuration.

In some embodiments, one or more impermeable layers can be disposed within the bellows cavity 131 and/or the bellows actuator 130 can comprise a material that is capable of holding a desired fluid (e.g., a fluid impermeable first internal layer 132 as discussed herein). The bellows actuator 130 can comprise a flexible, elastic, or deformable material that is operable to expand and contract when the bellows actuators 130 are inflated or deflated as described herein. In some embodiments, the bellows actuator 130 can be biased toward a deflated configuration such that the bellows actuator 130 is elastic and tends to return to the deflated configuration when not inflated. Additionally, although bellows actuator 130 shown herein are configured to expand and/or extend when inflated with fluid, in some embodiments, bellows actuator 130 can be configured to shorten and/or retract when inflated with fluid in some examples. Also, the term “bellows” as used herein should not be construed to be limiting in any way. For example, the term “bellows” as used herein should not be construed to require elements such as convolutions or other such features (although convoluted bellows actuator 130 can be present in some embodiments). As discussed herein, bellows actuator 130 can take on various suitable shapes, sizes, proportions and the like.

The bellows actuator 130 can vary significantly across various embodiments, so the present examples should not be construed to be limiting. One preferred embodiment of a bellows actuator 130 includes fabric-based pneumatic actuator configured such that it provides knee extension torque as discussed herein. Variants of this embodiment can exist to tailor the actuator to provide the desired performance characteristics of the actuators such as a fabric actuator that is not of a uniform cross-section. Other embodiments can use an electro-mechanical actuator configured to provide flexion and extension torques at the knee instead of or in addition to a fluidic bellows actuator 130. Various embodiments can include but are not limited to designs that incorporate combinations of electromechanical, hydraulic, pneumatic, electro-magnetic, or electro-static for positive power or negative power assistance of extension or flexion of a lower extremity joint.

The bellows actuator 130 can also be located in a variety of locations as required by the specific design. One embodiment places the bellows actuator 130 of a powered knee brace component located in line with the axis of the knee joint and positioned parallel to the joint itself. Various embodiments include but are not limited to, actuators configured in series with the joint, actuators configured anterior to the joint, and actuators configured to rest around the joint.

Various embodiments of the bellows actuator 130 can include secondary features that augment the operation of the actuation. One such embodiment is the inclusion of user-adjustable mechanical hard end stops to limit the allowable range of motion to the bellows actuator 130. Various embodiments can include but are not limited to the following extension features: the inclusion of flexible end stops, the inclusion of an electromechanical brake, the inclusion of an electro-magnetic brake, the inclusion of a magnetic brake, the inclusion of a mechanical disengage switch to mechanically decouple the joint from the actuator, or the inclusion of a quick release to allow for quick changing of actuator components.

In various embodiments, the bellows actuator 130 can comprise a bellows and/or bellows system as described in related U.S. patent application Ser. No. 14/064,071 filed Oct. 25, 2013, which issued as U.S. Pat. No. 9,821,475; as described in U.S. patent application Ser. No. 14/064,072 filed Oct. 25, 2013; as described in U.S. patent application Ser. No. 15/823,523 filed Nov. 27, 2017; or as described in U.S. patent application Ser. No. 15/472,740 filed Mar. 29, 2017.

In some applications, the design of the fluidic actuator unit 110 can be adjusted to expand its capabilities. One example of such a modification can be made to tailor the torque profile of a rotary configuration of the fluidic actuator unit 110 such that the torque changes as a function of the angle of the joint structure 125. To accomplish this in some examples, the cross-section of the bellows actuator 130 can be manipulated to enforce a desired torque profile of the overall fluidic actuator unit 110. In one embodiment, the diameter of the bellows actuator 130 can be reduced at a longitudinal center of the bellows actuator 130 to reduce the overall force capabilities at the full extension of the bellows actuator 130. In yet another embodiment, the cross-sectional areas of the bellows actuator 130 can be modified to induce a desired buckling behavior such that the bellows actuator 130 does not get into an undesirable configuration. In an example embodiment, the end configurations of the bellows actuator 130 of a rotary configuration can have the area of the ends reduced slightly from the nominal diameter to provide for the end portions of the bellows actuator 130 to buckle under loading until the actuator unit 110 extends beyond a predetermined joint angle, at which point the smaller diameter end portion of the bellows actuator 130 would begin to inflate.

In other embodiments, this same capability can be developed by modifying the behavior of the constraining ribs 135. In an example embodiment, using the same example bellows actuator 130 as discussed in the previous embodiment, two constraining ribs 135 can fixed to such bellows actuator 130 at evenly distributed locations along the length of the bellows actuator 130. In some examples, a goal of resisting a partially inflated buckling can be combated by allowing the bellows actuator 130 to close in a controlled manner as the actuator unit 110 closes. The constraining ribs 135 can be allowed to get closer to the joint structure 125 but not closer to each other until they have bottomed out against the joint structure 125. This can allow the center portion of the bellows actuator 130 to remain in a fully inflated state which can be the strongest configuration of the bellows actuator 130 in some examples.

In further embodiments, it can be desirable to optimize the fiber angle of the individual braid or weave of the bellows actuator 130 in order to tailor specific performance characteristics of the bellows actuator 130 (e.g., in an example where a bellows actuator 130 includes inextensibility provided by a braided or woven fabric). In other embodiments, the geometry of the bellows actuator 130 of the actuator unit 110 can be manipulated to allow the robotic exoskeleton system 100 to operate with different characteristics. Example methods for such modification can include but are not limited to the following: the use of smart materials on the bellows actuator 130 to manipulate the mechanical behavior of the bellows actuator 130 on command; or the mechanical modification of the geometry of the bellows actuator 130 through means such as shortening the operating length and/or reducing the cross-sectional area of the bellows actuator 130.

In further examples, a fluidic actuator unit 110 can comprise a single bellows actuator 130 or a combination of multiple bellows actuator 130, each with its own composition, structure, and geometry. For example, some embodiments can include multiple bellows actuator 130 disposed in parallel or concentrically on the same joint assembly 125 that can be engaged as needed. In one example embodiment, a joint assembly 125 can be configured to have two bellows actuator 130 disposed in parallel directly next to each other. The exoskeleton system 100 can selectively choose to engage each bellows actuator 130 as needed to allow for various amounts of force to be output by the same fluidic actuator unit 110 in a desirable mechanical configuration.

In further embodiments, a fluidic actuator unit 110 can include various suitable sensors to measure mechanical properties of the bellows actuator 130 or other portions of the fluidic actuator unit 110 that can be used to directly or indirectly estimate pressure, force, or strain in the bellows actuator 130 or other portions of the fluidic actuator unit 110. In some examples, sensors located at the fluidic actuator unit 110 can be desirable due to the difficulty in some embodiments associated with the integration of certain sensors into a desirable mechanical configuration while others may be more suitable. Such sensors at the fluidic actuator unit 110 can be operably connected to the exoskeleton device 1210 (see FIG. 12) and the exoskeleton device 1210 can use data from such sensors at the fluidic actuator unit 110 to control the exoskeleton system 100.

As discussed herein, various suitable exoskeleton systems 100 can be used in various suitable ways and for various suitable applications. However, such examples should not be construed to be limiting on the wide variety of exoskeleton systems 100 or portions thereof that are within the scope and spirit of the present disclosure. Accordingly, exoskeleton systems 100 that are more or less complex than the examples of FIGS. 1-5 are within the scope of the present disclosure.

Additionally, while various examples relate to an exoskeleton system 100 associated with the legs or lower body of a user, further examples can be related to any suitable portion of a user body including the torso, arms, head, legs, or the like. Also, while various examples relate to exoskeletons, it should be clear that the present disclosure can be applied to other similar types of technology, including prosthetics, body implants, robots, or the like. Further, while some examples can relate to human users, other examples can relate to animal users, robot users, various forms of machinery, or the like.

The described embodiments are susceptible to various modifications and alternative forms, and specific examples thereof have been shown by way of example in the drawings and are herein described in detail. It should be understood, however, that the described embodiments are not to be limited to the particular forms or methods disclosed, but to the contrary, the present disclosure is to cover all modifications, equivalents, and alternatives. Additionally, elements of a given embodiment should not be construed to be applicable to only that example embodiment and therefore elements of one example embodiment can be applicable to other embodiments. Additionally, elements that are specifically shown in example embodiments should be construed to cover embodiments that comprise, consist essentially of, or consist of such elements, or such elements can be explicitly absent from further embodiments. Accordingly, the recitation of an element being present in one example should be construed to support some embodiments where such an element is explicitly absent.

What is claimed is:

1. A method of operating an exoskeleton system, the method comprising:
coupling the exoskeleton system to a user, the exoskeleton system comprising:
a left and right leg actuator unit respectively coupled to a left and right leg of the user, the left and right leg actuator units each including:
an upper arm and a lower arm that are rotatably coupled via a joint, the joint positioned at a knee of the user with the upper arm coupled about an upper leg portion of the user above the knee and with the lower arm coupled about a lower leg portion of the user below the knee, and
a fluidic bellows actuator that extends between the upper arm and lower arm, and
a plurality of sensors that include:
a first and second rotary encoder respectively associated with the joints of the left and right leg actuator units, the first and second rotary encoders configured to measure respective joint angles of the left and right leg actuator units,
a GPS sensor, and
one or more accelerometer,
an exoskeleton device that includes:
a fluidic system, and
a processor and memory, the memory storing instructions, that when executed by the processor, are configured to control the fluidic system to introduce fluid to the fluidic bellows actuators of the left and right leg actuator units; and
obtaining, at the exoskeleton device, sensor data from the plurality of sensors;
determining, by the exoskeleton device based at least in part on a first portion of the sensor data, a plurality of states of the exoskeleton system including respective joint angles of the left and right leg actuator units;
determining, by the exoskeleton device based at least in part on a second portion of the sensor data, at least one state of the user wearing the exoskeleton system;
determining, by the exoskeleton device based at least in part on a third portion of the sensor data, at least one state of a location where the user and exoskeleton system are located;
determining, by the exoskeleton device, a response based at least in part on the determined plurality of states of the exoskeleton system, the determined at least one state of the user wearing the exoskeleton system, and the determined at least one state of the location where the user and the exoskeleton system are located; and
generating the response by the exoskeleton device at least controlling the fluidic system to introduce the fluid to the fluidic bellows actuators of the left and right leg actuator units to generate the response.

2. The method of claim 1, wherein the response further includes generating an alert that is presented on an external device that is wirelessly operably connected to the exoskeleton device.

3. The method of claim 1, wherein the determined at least one state of the user wearing the exoskeleton system corresponds to at least one of:
undesirable chemical, radiological or biological exposure to the user,
injury to the user,
fatigue of the user,
illness of the user,
stress level of the user, or
consciousness level of the user.

4. The method of claim 1, wherein one or both of the determined at least one state of the user wearing the exoskeleton system and the determined plurality of states of the exoskeleton system correspond to determining that the user and/or the exoskeleton system are in freefall.

5. The method of claim 1, wherein the determined at least one state of the location where the user and the exoskeleton system are located corresponds to one or more of:
current weather at the location where the user and the exoskeleton system are located;
a weather forecast at the location where the user and the exoskeleton system are located; or
a terrain of the location where the user and the exoskeleton system are located.

6. The method of claim 1, wherein the determined at least one state of the user wearing the exoskeleton system includes a determination that the user has exited a vehicle, the determination that the user has exited the vehicle being based at least in part on:
a previous determination that the user was riding in or on the vehicle, made based at least in part on a previous set of sensor data from the plurality of sensors, and a previous set of sensor data obtained from the vehicle; and
a current set of sensor data obtained from the vehicle and the sensor data from the plurality of sensors.

7. The method of claim 1, further comprising:
providing a prompt to the user via an interface that presents an indication of one or more of:
at least one of the determined plurality of states of the exoskeleton system,
the determined at least one state of the user wearing the exoskeleton system, or
the determined at least one state of the location where the user and the exoskeleton system are located;
providing a feedback mechanism, via the prompt to the user via the interface for the user to evaluate the indicated one or more of:
the at least one of the determined plurality of states of the exoskeleton system,
the determined at least one state of the user wearing the exoskeleton system, or
the determined at least one state of the location where the user and the exoskeleton system are located;
obtaining a feedback indication in response to the prompt; and
improving a classification program, an inference program, or an operating program of the exoskeleton system based at least in part on the feedback indication.

8. The method of claim 1, further comprising:
providing an alert to the user via an interface regarding a fitness test, the alert including instructions for performing one or more actions of the fitness test;
obtaining a set of fitness test sensor data generated during the user performing the one or more actions of the fitness test, the set of fitness test sensor data comprising the sensor data from the plurality of sensors;
determining a current ability level of the user based at least in part on the set of fitness test sensor data; and
comparing the current ability level of the user to a baseline ability level of the user; and
wherein determining the response is further based at least in part on the comparison of the current ability level of the user to the baseline ability level of the user.

9. The method of claim 1, further comprising:
obtaining, via a wireless network, a set of sensor data from a plurality of exoskeleton systems that are proximate to, but separate from, the exoskeleton system;
wherein determining the at least one state of the user wearing the exoskeleton system is further based at least in part on a first portion of the set of sensor data; and
wherein determining, the at least one state of the location where the user and the exoskeleton system are located is further based at least in part on a second portion of the set of sensor data.

10. A method of operating an exoskeleton system, the method comprising:
obtaining, at an exoskeleton device, sensor data from one or more sensors;
determining, by the exoskeleton device based at least in part on the sensor data, one or more states, including one or more of:
at least one state of the exoskeleton system;
at least one state of a user wearing the exoskeleton system; and
at least one state of a location where the user and exoskeleton system are located;
determining, by the exoskeleton device, a response based at least in part on the determined one or more states; and
generating the response by the exoskeleton device causing actuation of the exoskeleton system.

11. The method of claim 10, wherein the exoskeleton system comprises:
one or more actuator units that includes a fluidic actuator;
a plurality of sensors that includes one or more of:
one or more encoder respectively associated with the one or more actuator units, the one or more encoders configured to measure a joint angle of the one or more actuator units,
a GPS sensor, or
one or more accelerometer.

12. The method of claim 10, wherein the determined at least one state of the user wearing the exoskeleton system corresponds to at least one of:
undesirable chemical, radiological or biological exposure to the user,
injury to the user,
fatigue of the user,
illness of the user,
stress level of the user, or
consciousness level of the user.

13. The method of claim 10, wherein one or both of the determined at least one state of the user wearing the exoskeleton system and the determined at least one state of the exoskeleton system correspond to determining that the user and/or the exoskeleton system are in freefall.

14. The method of claim 10, wherein the determined at least one state of the location where the user and the exoskeleton system are located corresponds to one or more of:
current weather at the location where the user and the exoskeleton system are located;
a weather forecast at the location where the user and the exoskeleton system are located; or
a terrain of the location where the user and the exoskeleton system are located.

15. The method of claim 10, wherein the determined at least one state of the user wearing the exoskeleton system includes a determination that the user has exited a vehicle, the determination that the user has exited the vehicle being based at least in part on:
a set of vehicle sensor data obtained from the vehicle and the sensor data from the one or more sensors.

16. The method of claim 10, further comprising:
providing a prompt via an interface that presents an indication of one or more of:
the determined at least one state of the exoskeleton system,
the determined at least one state of the user wearing the exoskeleton system, or
the determined at least one state of the location where the user and the exoskeleton system are located;
providing a feedback mechanism via the interface to evaluate the indicated one or more of:
the determined at least one state of the exoskeleton system,
the determined at least one state of the user wearing the exoskeleton system, or
the determined at least one state of the location where the user and the exoskeleton system are located; and
obtaining a feedback indication in response to the prompt.

17. The method of claim 10, further comprising:
providing an alert via an interface regarding a fitness test, the alert including instructions for performing one or more actions of the fitness test;
obtaining a set of fitness test sensor data generated during the user performing the one or more actions of the fitness test, the set of fitness test sensor data comprising the sensor data from the one or more sensors; and
determining an ability level of the user based at least in part on the set of fitness test sensor data; and
wherein determining the response is further based at least in part on the determined ability level of the user.

18. The method of claim 10, further comprising:
obtaining a set of sensor data from a plurality of exoskeleton systems that are proximate to the exoskeleton system; and
wherein determining the at least one state of the user wearing the exoskeleton system is further based at least in part on a first portion of the set of sensor data; or
wherein determining, the at least one state of the location where the user and the exoskeleton system are located is further based at least in part on a second portion of the set of sensor data.
